(19) United States
(12) Patent Application Publication
(10) Pub. No.: US 2005/0014769 A1
(43) Pub. Date: Jan. 20, 2005

(54) Use of endothelin receptor antogonists for the treatment of tumour diseases

(76) Inventors: Mathias Osswald, Alsbach-Hahnlein (DE); Dieter Dorsch, Ober-Ramstadt (DE); Werner Mederski, Zwingenberg (DE); Christiane Amendt, MAinz (DE); Matthias Grell, Darmstadt (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/495,108

(22) PCT Filed: Oct. 10, 2002
(86) PCT No.: PCT/EP02/11350

(30) Foreign Application Priority Data
Nov. 9, 2001 (DE) 101 55 076.6

(51) Int. Cl. A61K 31/519; A61K 31/4745

(52) U.S. Cl. 514/260.1; 514/301; 514/302

(57) ABSTRACT

Use of endothelin receptor antagonists for the preparation of a medicament for the treatment of tumour diseases.

The invention relates to the use of endothelin receptor antagonists selected from the group consisting of
a) the compounds of the formula I described in EP 0733626 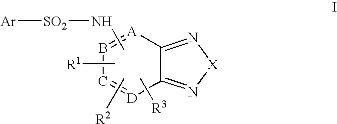
   in which
-A=B—C=D- is a —CH═CH—CH═CH— group in which 1 or 2 CH has (have) been replaced by N,
Ar is Ph or naphthyl, each of which is unsubstituted or monosubstituted, disubstituted or trisubstituted by H, Hal, A, alkenyl having up to 6 carbon atoms, Ph, OPh, NO₂, NR⁴R⁵, NHCOR⁴, CF₃, OCF₃, CN, OR⁴, COOR⁴, (CH₂)ₙCOOR⁴, (CH₂)ₙNR⁴R⁵, —N═C═O or NHCONR⁴R⁵,
R¹, R²
and R³ are each, independently of one another, absent, H, Hal, A, CF₃, NO₂, NR⁴R⁵, CN, COOR⁴ or NHCOR⁴,
R⁴ and R⁵ are each, independently of one another, H or A, or together are alternatively —CH₂—(CH₂)ₙ—CH₂—,
A is alkyl having from 1 to 6 carbon atoms,
Ph is phenyl,
X is O or S,
Hal is F, Cl, Br or I,
n is 1, 2 or 3,
and salts thereof;
b) the compounds of the formula I described in EP 0758650 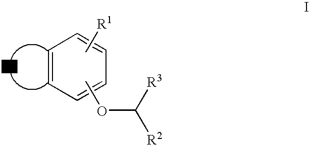
   in which
X is a saturated, fully unsaturated or partially unsaturated 3- to 4-membered alkylene chain, in which from 1 to 3 carbon atoms may be replaced by N and/or 1 or 2 carbon atoms may be replaced by 1-2 O atoms and/or 1-2 S atoms, but where at most up to 3 carbon atoms are replaced and where, in addition, a monosubstitution, disubstitution or trisubstitution of the alkylene chain and/or of a nitrogen located therein by A, R⁸ and/or NR⁴R^{4′} may occur, and where, furthermore, one CH₂ group in the alkylene chain may also be replaced by a C═O group,
A is alkyl having 1-6 carbon atoms, in which one or two CH₂ groups may be replaced by O or S atoms or by —CR⁴═CR^{4′}— groups and in addition 1-7 H atoms may be replaced by F,
R¹ is H or A,
R² is COOR⁴, CN, 1H-tetrazol-5-yl or CONHSO₂R⁸,
R³ is Ar,
R⁴ and R^{4′} are each, independently of one another, H, alkyl having from 1 to 6 carbon atoms or benzyl,
Ar is phenyl or naphthyl, each of which is unsubstituted or monosubstituted, disubstituted or trisubstituted by R⁵, R⁶ or R⁷, or a 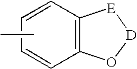
   group which is unsubstituted or monosubstituted or disubstituted in the phenyl moiety by R⁵ or R⁶,
R⁵, R ⁶
and R⁷ are each, independently of one another, R⁴, OR⁴, Hal, CF₃, OCF₃, OCHF₂, OCH₂F, NO₂, NR⁴R^{4′}, NHCOR⁴, CN, NHSO₂R⁴ COOR⁴, COR⁴, CONHSO₂R⁸, O(CH₂)ₙR², OPh, O(CH₂)ₙOR⁴ or S(O)ₘR⁴,
R⁸ is phenyl or naphthyl, each of which is unsubstituted or monosubstituted, disubstituted or trisubstituted by A, OR¹, NR⁴R^{4′} or Hal,
E is CH₂ or O,
D is carbonyl or [C(R⁴R^{4′})]ₙ,
Hal is F, Cl, Br or I,
m is 0, 1 or 2,
n is1 or 2,
and salts thereof;
c) the compounds of the formula I described in EP 0755934 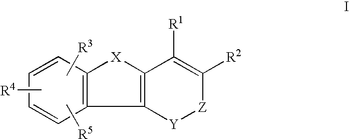
   in which
—Y-Z- is —NR⁷—CO—, —N═C(OR⁷)— or —N═CR⁸—,
R¹ is Ar,
R² is COOR⁶, CN, 1H-tetrazol-5-yl or CONHSO₂Ar,
R³, R⁴
and R⁵ are each, independently of one another, R⁶, OR⁶, S(O)ₘR⁶, Hal, NO₂, NR⁶R^{6′}, NHCOR⁶, NHSO₂R⁶, OCOR⁶, COOR⁶ or CN,
R⁶ and R^{6′} are each, independently of one another, H, alkyl having from 1 to 6 carbon atoms, benzyl or phenyl,
R⁷ is (CH₂)ₙAr,
R⁸ is Ar or OAr,
Ar is phenyl which is unsubstituted or monosubstituted, disubstituted or trisubstituted by R⁹, R¹⁰ or R¹¹, or unsubstituted naphthyl, or a 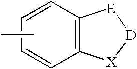
   group which is unsubstituted or monosubstituted or disubstituted in the phenyl moiety by R⁹ or R¹⁰, or a 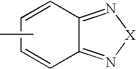
   group which is unsubstituted or monosubstituted or disubstituted in the cyclohexadienyl moiety by R⁹ or R¹⁰,
R⁹, R¹⁰
and R¹¹ are each, independently of one another, R⁶, OR⁶, Hal, CF₃, OCF₃, OCHF₂, OCH₂F, NO₂, NR⁶R^{6′}, NHCOR⁶, CN, NHSO₂R⁶, COOR⁶, COR⁶, CONHSO₂Ar, O(CH₂)ₙR², O(CH₂)ₙOR⁶ or S(O)ₘR⁶,
E is CH₂, S or O,
D is carbonyl or [C(R⁶R^{6′})]ₙ,
Hal is F, Cl, Br or I,
X is O or S,
m is0, 1 or 2,
n is 1 or 2,
and salts thereof;
d) the compounds of the formula I described in EP 0757039 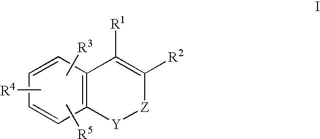
   in which
—Y-Z- is —NR⁷—CO—, —N═C(OR— or —N═CR⁸—,
R¹ is Ar,
R² is COOR⁶, (CH₂)ₙCOOR , CN, 1H-tetrazol-5-yl or CONHSO₂Ar,
R³, R⁴
and R⁵ are each, independently of one another, R⁶, OR⁶, S(O)ₘR⁶, Hal, NO₂, NR⁶R^{6′}, NHCOR⁶, NHSO₂R⁶, OCOR⁶, COR⁶, COOR6 or CN, where R³ and R⁴ together can alternatively be an O(CH₂)ₙO group,
R⁶ and R^{6′} are each, independently of one another, H, alkyl having from 1 to 6 carbon atoms, benzyl or phenyl,
R⁷ is (CH₂)ₙAr,
R⁸ is Ar or OAr,
Ar is phenyl which is unsubstituted or monosubstituted, disubstituted or trisubstituted by R⁹, R¹⁰ or R¹¹, or unsubstituted naphthyl, or a 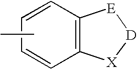
   group which is unsubstituted or monosubstituted or disubstituted in the phenyl moiety by R⁹ or R¹⁰, or a 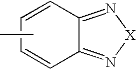
   group which is unsubstituted or monosubstituted or disubstituted in the cyclohexadienyl moiety by R⁹ or R¹⁰,
R⁹, R¹⁰
and R¹¹ are each, independently of one another, R⁶, OR⁶, Hal, CF₃, OCF₃, OCHF₂, OCH₂F, NO₂, NR⁶R^{6′}, NHCOR⁶, CN, NHSO₂R⁶, COOR⁶, COR⁶, CONHSO₂Ar, O(CH₂)ₙR², O(CH₂)ₙOR⁶ or S(O)ₘR⁶,
E is CH₂, S or O,
D is carbonyl or [C(R⁶R^{6′})]ₙ,
X is O or S,
Hal is F, Cl, Br or I,
m is0, 1 or 2,
n is 1, 2or 3,
and salts thereof;
e) the compounds of the formula I described in EP 0796250 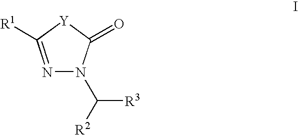
   in which
Y is —C(R⁴R^{4′})—C(R⁴R^{4′})—, —CR⁴═CR^{4′}— or —C(R⁴R^{4′})—S—,
R¹ is Het, Ar, R³ or R⁴,
R² is Ar or
   a 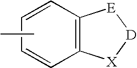
      group which is unsubstituted or monosubstituted or disubstituted in the phenyl moiety by A, R³, OR⁴, NH₂, NHA, NA₂, NO₂, CN, Hal, NHCOR⁴, NHSO₂R⁴, COOR⁴, COR⁴, CONHSO₂R⁶, O(CH₂)ₙR³, OPh, O(CH₂)ₙOR⁴ or S(O)ₘR⁴, or a 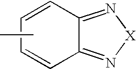
      group which is unsubstituted or monosubstituted or disubstituted in the cyclohexadienyl moiety by A, R³, OR⁴, NH₂, NHA, NA₂, NO₂, CN, Hal, NHCOR⁴, NHSO₂R⁴, COOR⁴, COR⁴, CONHSO₂R⁶, O(CH₂)ₙR³, OPh, O(CH₂)ₙOR⁴ or S(O)ₘR⁴,
R³ is CN, COOH, COOA, CONHSO₂R⁵ or 1H-tetrazol-5-yl,
R⁴ and R^{4′} are each, independently of one another, H, A, or phenyl or benzyl, each of which is unsubstituted or monosubstituted by alkoxy,
R⁵ is A or Ar,
R⁶ is phenyl or naphthyl, each of which is unsubstituted or monosubstituted, disubstituted or trisubstituted by A, OR⁵, NH₂, NHA, NA₂, NO₂, CN or Hal,
A is alkyl having 1-6 carbon atoms, in which one or two CH₂ groups may be replaced by O or S atoms or by —CR⁴═CR^{4,}— groups and in addition 1-7 H atoms may be replaced by F,
   or benzyl,
Ar is phenyl or naphthyl, each of which is unsubstituted or monosubstituted, disubstituted or trisubstituted by A, OR⁴, NH₂, NHA, NA₂, NO₂, CN, Hal, NHCOR⁴, NHSO₂R⁴, COOR⁴, COR⁴, CONHSO₂R⁶, O(CH₂)ₙR³, OPh, O(CH₂)ₙOR⁴ or S(O)ₘR⁴,
Het is a monocyclic or bicyclic saturated, unsaturated or aromatic heterocyclic radical having from 1 to 4 N, O and/or S atoms, bonded via N or C, which may be unsubstituted or monosubstituted, disubstituted or trisubstituted by Hal, A, R³, NH₂, NHA, NA₂, CN, NO₂ and/or carbonyl oxygen,
D is carbonyl or [C(R⁴R^{4′})]ₙ,
E is CH₂, S or O,
Hal is F, Cl, Br or I,
X is O or S,
m is 0, 1 or 2,
n is 1 or 2,
and salts thereof;
f) the compounds of the formula I described in WO 9719077 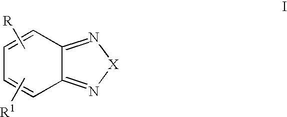
   in which
R is 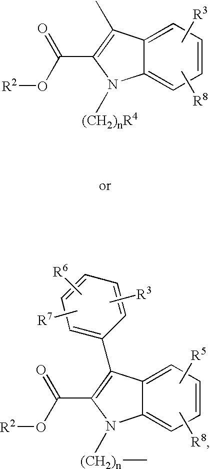
X is O or S,
R¹ is H, Hal, OH, OA, A, alkylene-O-A, NO₂, NH₂, NH-acyl, SO₂NH₂, SO₃-A, SO₂NHA, CN or formyl,
R² is H or A,
R³, R⁵, R⁶,
R⁷ and R⁸ are each, independently of one another, H, Hal, OH, OA, O-alkylene-R⁴, A, S-A, NO₂, NH₂, NHA, NA₂, NH-acyl, NHSO₂A, NHSO₂R⁴, NASO₂A, NASO₂—R⁴, NH(CO)NH₂, NH(CO)NHA, formyl, NH(CO)NH-phenyl, NHCOOA, NA-acyl, NHR⁴, NHCOOR⁴, NHCOO-benzyl, NHSO₂-benzyl, NHCOO-alkylene-OA, NH(CO)NA₂, N-piperidinyl-CO—NH, N-pyrrolidinyl-CONH, O(CH₂)ₙCOOR², O(CH₂)ₙOR², CH₂OH or CH₂OA,
R³ and R⁶ together are alternatively —O—CH₂—O—, —O—CH₂—CH₂—O—, —O—CH₂—CH₂—, —O—CF₂—O— or —O—CF₂—CF₂—O—,
R⁴ is phenyl which is unsubstituted or monosubstituted or polysubstituted by R³ and/or R⁶,
A is alkyl having 1-6 carbon atoms,
Hal is fluorine, chlorine, bromine or iodine,
n is 1 or 2,
and salts thereof;
g) the compounds of the formula I described in WO 9730982 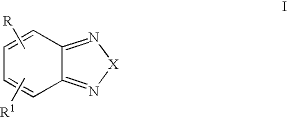
   in which
R is 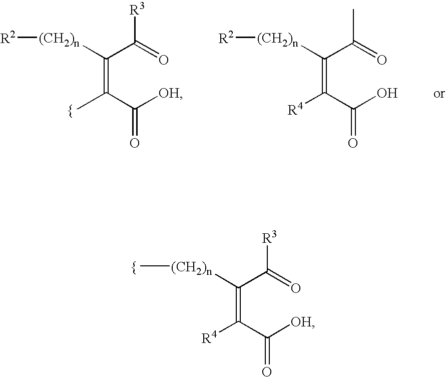
X is O or S,
R¹ is H, Hal, OH, OA, A, alkylene-O-A, NO₂, NH₂, NH-acyl, SO₂NH₂, SO₃-A, SO₂NHA, CN or formyl,
R², R³
and R⁴ are each, independently of one another, a phenyl group which is unsubstituted or monosubstituted or polysubstituted by Hal, OH, OA, O-alkylene-R⁵, A, S-A, SOA, SO₂A, SOR⁵, SO₂R⁵, NO₂, NH₂, NHA, NA₂, NH-acyl, NHSO₂A, NHSO₂R⁵, NASO₂A, NASO₂—R⁵, NH(CO)NH₂, NH(CO)NHA, formyl, NH(CO)NHR⁵, NHCOOA, NA-acyl, NHCOOCH₂R⁵, NHSO₂CH₂R⁵, NHCOO-alkylene-OA, NH(CO)NA₂, 1 -piperidinyl-CO—NH, 1-pyrrolidinyl-CONH, O(CH₂)ₙCOOA, O(CH₂)ₙCOOH, O(CH₂)ₙOH, O(CH₂)ₙOA, CH₂OH, CH₂OA, COOH, COOA, CH₂COOH or CH₂COOA,
   a 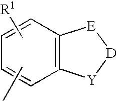
      group or a 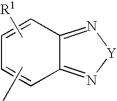
      group, where R² is additionally A or cycloalkyl,
R⁵ is a phenyl group which is unsubstituted or monosubstituted or polysubstituted by Hal, OH, OA, A, S-A, NO₂, NH₂, NHA, NA₂, NH-acyl, NHSO₂A, NASO₂A, NH(CO)NH₂, NH(CO)NHA, formyl, NHCOOA, NA-acyl, NHCOO-alkylene-OA, NH(CO)NA₂, N-piperidinyl-CO—NH, N-pyrrolidinyl-CONH, O(CH₂)ₙCOOA, O(CH₂)ₙCOOH, O(CH₂)ₙOH, O(CH₂)ₙOA, CH₂OH, CH₂OA, COOH, COOA, CH₂COOH or CH₂COOA,
A is alkyl having 1-6 carbon atoms, in which one or two CH₂ groups may be replaced by O or S atoms or by —CR⁶═CR^{6′}— groups and/or 1-7 H atoms may be replaced by F,
D is carbonyl or [C(R⁶R^{6′})]ₘ,
E is CH₂, S or O,
Y is O or S,
R⁶ and R^{6′} are each, independently of one another, H, F or A,
Hal is fluorine, chlorine, bromine or iodine,
n is 1 or 2, and
m is 1 or 2,
or a tautomeric cyclised form, and the (E)-isomers and the salts of all isomers;
h) the compounds of the formula I described in WO 9730996 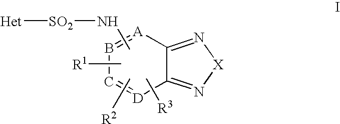
   in which
-A=B—C=D- is a —CH═CH—CH═CH— group, in which, in addition, 1 or 2 CH may be replaced by N,
Het is a monocyclic or bicyclic saturated, unsaturated or aromatic heterocyclic radical having from 1 to 4 N, O and/or S atoms which is unsubstituted or substituted by -Z-R⁶,
R¹, R²
and R³ are each, independently of one another, absent, H, Hal, A, CF₃, NO₂, NR⁴R⁵, CN, COOR⁴ or NHCOR⁴,
R⁴ and R⁵ are each, independently of one another, H or A, or together are alternatively —CH₂—(CH₂)ₙ—CH₂—,
R⁶ is a phenyl radical, a benzothiadiazol-5-yl radical or a benzoxadiazol-5-yl radical, each of which is unsubstituted or monosubstituted, disubstituted or trisubstituted by R⁷, R⁸ and/or R⁹,
R⁷, R⁸
and R⁹ are each, independently of one another, A, O-A, CN, COOH, COOA, Hal, formyl or —CO-A, and R⁷ and R⁸ together are alternatively —O—(CH₂)ₘ—O—,
A is alkyl having from 1 to 6 carbon atoms,
X is O or S,
Z is —CO—, —CONH—, —CO—(CH₂)ₙ—, —CH═CH—, —(CH₂)ₙ—, —CONHCO—, —NHCONH—, —NHCOO—, —O—CONH—, —CO—O— or —O—CO—,
Hal is F, Cl, Br or I,
m is 1 or 2, and
n is1, 2 or 3,
and salts thereof;
i) the compounds of the formula I described in DE 19609597 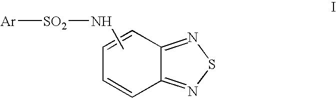
   in which
Ar is naphthyl which is monosubstituted by NH₂, NHA or NA₂, and
A is alkyl having from 1 to 6 carbon atoms,
and physiologically acceptable salts thereof;
j) the compounds of the formula I described in DE 19612101 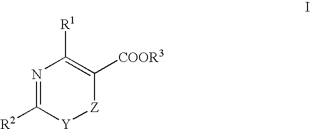
   in which
—Y-Z- is —NR⁴—CO or —N═CR⁵—,
R¹ is Ar,
R² is H, alkyl having 1-6 carbon atoms which is unsubstituted or monosubstituted, disubstituted or trisubstituted by OR³ or Hal, or (CH₂)ₘPh or (CH₂)ₘ-cycloalkyl, each of which is unsubstituted or monosubstituted, disubstituted or trisubstituted by R³, OR³ or Hal,
R³ and R^{3′} are each, independently of one another, H, alkyl having 1-6 carbon atoms or benzyl,
R⁴ is CH₂Ar,
R⁵ is OCH₂Ar,
Ar is phenyl which is unsubstituted or monosubstituted, disubstituted or trisubstituted by R⁶, R⁷ or R⁸, or a 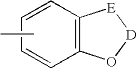
   group which is unsubstituted or monosubstituted in the phenyl moiety by R⁶, or a 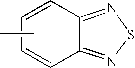
   group which is unsubstituted or monosubstituted in the cyclo-hexadienyl moiety by R⁶,
E is CH₂ or O,
D is carbonyl or (CH₂)ₙ,
E and D together are alternatively CH═CR⁹,
R⁶ and R^{6′} are each, independently of one another, R³, OR³ or Hal,
R⁷ is R³, OR³, Hal, NO₂, NH₂, NHR³, NR³R^{3′}, NHCOR³, COOR³, O(CH₂)ₙR³ or O(CH₂)ₙOR³,
R⁸ is Ph which is unsubstituted or monosubstituted, disubstituted or trisubstituted by R³, OR³, Hal, NO₂, NH₂, NHR⁶, NR⁶R^{6′}, NHCOR³ or COOR³,
R⁹ is H, OH, CH₂OH or COOR³,
Hal is F, Cl, Br or I,
Ph is phenyl,
m is 0 or 1,
n is 1 or 2,
and salts thereof;
k) the compounds of the formula I described in WO 9827091 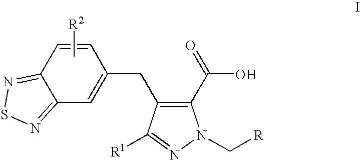
   in which
R is phenyl which is unsubstituted or monosubstituted, disubstituted or trisubstituted by R³, R⁴ or R⁵ or is 2,1,3-benzothiadiazolyl which is unsubstituted or monosubstituted by R²,
R¹ is A, in which 1-7 H atoms may be replaced by F, or is —S-A or —O-A, or phenyl or -alkylene-phenyl, each of which is unsubstituted or monosubstituted by R³, or thienyl which is unsubstituted or monosubstituted by R³,
R² is A, F, Cl, Br or —O-A,
R³, R⁴
and R⁵ are each, independently of one another, A, —O-A, —S-A, —O-alkylene-COOH, -alkylene-COOH or COOH,
R³ and R⁴ together are alternatively —O—CH₂—O—, and
A is alkyl having 1-7 carbon atoms,
and salts thereof;
I) the compounds of the formula I described in WO 9827077 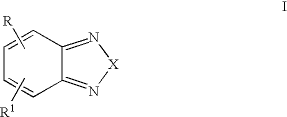
   in which
R is 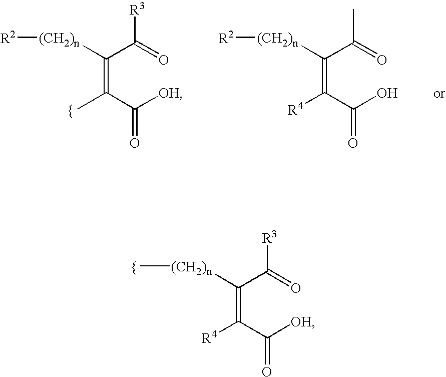
X is O or S,
R¹ is H, Hal, OH, OA, A, alkylene-O-A, NO₂, NH₂, NH-acyl, SO₂NH₂, SO₃-A, SO₂NHA, CN or formyl,
R², R³
and R⁴ are each, independently of one another, a phenyl group which is unsubstituted or monosubstituted or polysubstituted by R⁷, where R² is additionally A or cycloalkyl,
   a 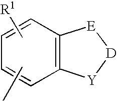
      group or a 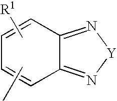
      group, with the proviso that at least one of the radicals R², R³ or R⁴ is an R⁸ radical which is unsubstituted or mono-substituted or polysubstituted by R⁷,
R⁵ is a phenyl group which is unsubstituted or monosubstituted or polysubstituted by Hal, OH, OA, A, S-A, NO₂, NH₂, NHA, NA₂, NH-acyl, NHSO₂A, NASO₂A, NH(CO)NH₂, NH(CO)NHA, formyl, NHCOOA, NA-acyl, NHCOO-alkylene-OA, NH(CO)NA₂, N-piperidinyl-CO—NH, N-pyrrolidinyl-CONH, O(CH₂)ₙCOOA, O(CH₂)ₙCOOH, O(CH₂)ₙOH, O(CH₂)ₙOA, CH₂OH, CH₂OA, COOH, COOA, CH₂COOH or CH₂COOA,
A is alkyl having 1-6 carbon atoms, in which one or two CH₂ groups may be replaced by O or S atoms or by —CR⁶═CR^{6′}— groups and/or 1-7 H atoms may be replaced by F,
D is carbonyl or [C(R⁶R^{6′})]ₘ,
E is CH₂, S or O,
Y is O or S,
R⁶ and R^{6′} are each, independently of one another, H, F or A,
R⁷ is Hal, OH, OA, O-alkylene-R⁵, A, S-A, S—OA, SO₂A, S—OR⁵, SO₂R⁵, NO₂, NH₂, NHA, NA₂, NH-acyl, NHSO₂A, NHSO₂R⁵, NASO₂A, NASO₂,R⁵, NH(CO)NH₂, NH(CO)NHA, formyl, NH(CO)NHR⁵, NHCOOA, NA-acyl, NHCOOCH₂R⁵, NHSO₂CH₂R⁵, NHCOO-alkylene-OA, NH(CO)NA₂, 1-piperidinyl-CO—NH, 1-pyrrolidinyl-CONH, O(CH₂)ₙCOOA, O(CH₂)ₙCOOH, O(CH₂)ₙOH, O(CH₂)ₙOA, CH₂OH, CH₂OA, COOH, COOA, CH₂COOH or CH₂COOA,
R⁸ is a 5-7 membered heterocyclic radical having 1-4 N, O and/or S atoms or
   a 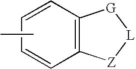
      group,
G and Z are each, independently of one another, —CH═, N, O or S,
L is —CH═, —CH═CH— or —CH₂—CH₂—CH₂—,
Hal is fluorine, chlorine, bromine or iodine,
n is 0, 1 or 2, and
m is 1 or 2,
or a tautomeric cyclised form, and the (E)-isomers and the salts of all isomers;
m) the compounds of the formula I described in WO 9841515 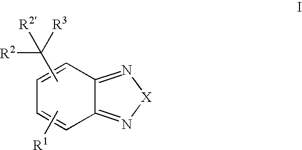
   in which
X is O or S,
R¹ is H, Hal, OH, OA, A, NO₂, NH₂, NHA, NAA′, NHCOR⁴, NHCOR⁶, NHSO₂R⁴, NHSO₂R⁶, S(O)ₘR⁶, SO₃H, SO₂NR⁴R^{4′} or formyl,
R² and R^{2′} are each, independently of one another, A, (CH₂)ₙAr, (CH₂)ₙHet, CH₂COAr, CH₂COHet or OAr,
R^{2′} is additionally also H,
R³ is COOR⁴, CN, 1H-tetrazol-5-yl or CONHSO₂R⁵,
R⁴ and R^{4′} are each, independently of one another, H or A,
R⁵ is A or Ar,
R⁶ is phenyl or naphthyl, each of which is unsubstituted or monosubstituted, disubstituted or trisubstituted by A, NH₂, NHA, NAA′, NO₂, CN or Hal,
R⁷ and R^{7′} are each, independently of one another, H or alkyl having 1-6 carbon atoms,
A and A′ are each, independently of one another, alkyl having 1-6 carbon atoms, in which one or two CH₂ groups may be replaced by O or S atoms or by —CR⁷═CR^{7′} groups and/or 1-7 H atoms may be replaced by F, or benzyl,
Ar is phenyl or naphthyl, each of which is unsubstituted or monosubstituted, disubstituted or trisubstituted by A, OR⁴, NH₂, NHA, NAA′, NO₂, CN, Hal, NHCOR⁴, NHCOR⁶, NHSO₂R⁴, NHSO₂R⁶, COOR⁴, OPh, CONH₂, CONHA, CONAA′, COR⁴, CONHSO₂R⁴, CONHSO₂R⁶, O(CH₂)ₙCOOR⁴, O(CH₂)ₙOR⁴, SO₃H, SO₂NR⁴R^{4′}, S(O)ₘR⁶ or S(O)ₘR⁴,
Het is a monocyclic or bicyclic saturated, unsaturated or aromatic heterocyclic radical having 1-4 N, O and/or S atoms, bonded via N or C, which may be unsubstituted or monosubstituted, disubstituted or trisubstituted by Hal, A, R³, NH₂, NHA, NAA′, NO₂ and/or ═O,
Hal is fluorine, chlorine, bromine or iodine,
m is 0, 1 or 2, and
n is 1 or 2,
where, if R² is CH₂COAr and R^{2′} is H, R³ is not COOA,
and salts thereof;
n) the compounds of the formula I described in WO 9841521 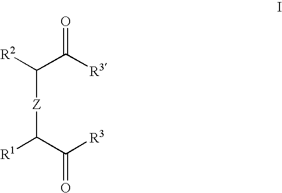
   in which
Z is a single or double bond,
R¹ is a 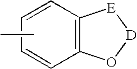
   group which is unsubstituted or monosubstituted in the phenyl moiety by R⁷, or a 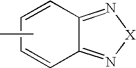
   group which is unsubstituted or monosubstituted in the cyclo-hexadienyl moiety by R⁷,
R² is A, Ar—(CH₂)ₘ, cycloalkyl-(CH₂)ₘ, Het-(CH₂)ₘ or R¹—(CH₂)ₘ,
R³ and R^{3′} are each, independently of one another, OR⁴, NHSO₂R⁵, NH₂, NHA or NAA′,
R³ and R^{3′} together are alternatively —O—, forming a cyclic anhydride,
R⁴ and R^{4′} are each, independently of one another, H or A,
R⁵ is A or Ar,
R⁶ is phenyl or naphthyl, each of which is unsubstituted or monosubstituted, disubstituted or trisubstituted by A, NH₂, NHA, NAA′, NO₂, CN or Hal,
R⁷ is A, COOR⁴, CN, 1H-tetrazol-5-yl, CONHSO₂R⁵, Hal, OR⁴, NO₂, NH₂, NHA, NAA′, NHCOR⁴, NHCOR⁶, NHSO₂R⁴, NHSO₂R⁶, S(O)ₖR⁴, S(O)ₖR⁶, SO₂NR⁴R^{4′} or formyl,
R⁸ and R^{8′} are each, independently of one another, H or alkyl having 1-6 carbon atoms,
E is CH₂ or O,
D is carbonyl or (CR⁴R^{4′})ₙ,
E and D together are alternatively CR⁴═R^{4′},
X is S or O,
A and A′ are each, independently of one another, alkyl having 1-6 carbon atoms, in which one or two CH₂ groups may be replaced by O or S atoms or by —CR⁸═CR^{8′}— groups and/or 1-7 H atoms may be replaced by F,
   or benzyl,
Ar is phenyl or naphthyl, each of which is unsubstituted or monosubstituted, disubstituted or trisubstituted by A, OR⁴, NH₂, NHA, NAA′, NO₂, CN, Hal, NHCOR⁴, NHCOR⁶, NHSO₂R⁴, NHSO₂R⁶, COOR⁴, OPh, CONH₂, CONHA, CONAA′, COR⁴, CONHSO₂R⁴, CONHSO₂R⁶, O(CH₂)ₙCOOR⁴, O(CH₂)ₙOR⁴, SO₂NR⁴R^{4′}, S(O)ₖR⁶ or S(O)ₖR⁴,
Het is a monocyclic or bicyclic saturated, unsaturated or aromatic heterocyclic radical having 1-4 N, O and/or S atoms, bonded via N or C, which may be unsubstituted or mono-substituted, disubstituted or trisubstituted by Hal, A, COOR⁴, CN, 1H-tetrazol-5-yl, CONHSO₂R⁵, NH₂, NHA, NAA′, NO₂ and/or ═O,
Hal is fluorine, chlorine, bromine or iodine,
k is 0, 1 or 2,
m is 0, 1 or 2, and
n is 1 or 2,
and the (Z)- and (E)-isomers and the salts of all isomers;
o) the compounds of the formula I described in WO 9842702 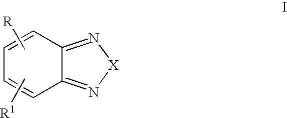
   in which
R is 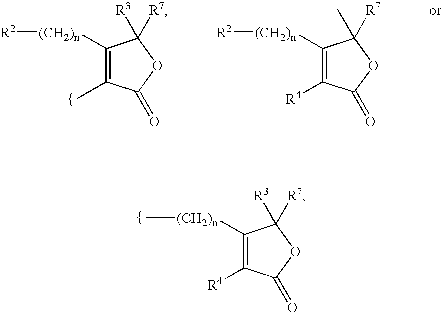
X and Y are each, independently of one another, O or S,
R¹ is H, Hal, OH, OA, A, alkylene-O-A, NO₂, NH₂, NH-acyl, SO₂NH₂, SO₂-A, SO₂NHA, CN or formyl,
R², R³
and R⁴ are each, independently of one another, a phenyl group which is unsubstituted or monosubstituted or polysubstituted by Hal, OH, OA, O-alkylene-R⁵, A, S-A, S—OA, SO₂A, S—OR⁵, SO₂R⁵, NO₂, NH₂, NHA, NA₂, NH-acyl, NHSO₂A, NHSO₂R⁵, NASO₂A, NASO₂—R⁵, NH(CO)NH₂, NH(CO)NHA, formyl, NH(CO)NHR⁵, NHCOOA, NA-acyl, NHCOOCH₂R⁵, NHSO₂CH₂R⁵, NHCOO-alkylene-OA, NH(CO)NA₂, 1-piperidinyl-CO—NH, 1-pyrrolidinyl-CONH, O(CH₂)ₙCOOA, O(CH₂)ₙCOOH, O(CH₂)ₙOH, O(CH₂)ₙOA, CH₂OH, CH₂OA, COOH, COOA, CH₂COOH or CH₂COOA,
   a 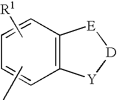
      group or a 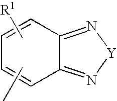
      group, where R² is additionally A or cycloalkyl,
R⁵ is a phenyl group which is unsubstituted or monosubstituted or polysubstituted by Hal, OH, OA, A, S-A, NO₂, NH₂, NHA, NA₂, NH-acyl, NHSO₂A, NASO₂A, NH(CO)NH₂, NH(CO)NHA, formyl, NHCOOA, NA-acyl, NHCOO-alkylene-OA, NH(CO)NA₂, N-piperidinyl-CO—NH, N-pyrrolidinyl-CONH, O(CH₂)ₙCOOA, O(CH₂)ₙCOOH, O(CH₂)ₙOH, O(CH₂)ₙOA, CH₂OH, CH₂OA, COOH, COOA, CH₂COOH or CH₂COOA,
A is alkyl having 1-6 carbon atoms, in which one or two CH₂ groups may be replaced by O or S atoms or by —CR⁶═CR^{6′}— groups and/or 1-7 H atoms may be replaced by F,
D is carbonyl or [C(R⁶R^{6′})]ₘ,
E is CH₂, S or O,
R⁶ and R^{6′} are each, independently of one another, H, F or A,
R⁷ is —O—C(═Y)—NH—R⁸,
R⁸ is alkyl having 1-10 carbon atoms which is unsubstituted or monosubstituted or disubstituted by R⁹ and in which 1-2 carbon atoms may be replaced by O and/or S and/or may be substituted by ═O,
   or
   cycloalkyl, in which 1-2 carbon atoms may be replaced by N, O and/or S
R⁹ is phenyl which is unsubstituted or monosubstituted or disubstituted by Hal,
   naphthyl, A-O—C(═O)— or Hal,
Hal is fluorine, chlorine, bromine or iodine,
n is 0, 1 or 2, and
m is 1 or 2,
and salts thereof;
p) the compounds of the formula I described in WO 9842709 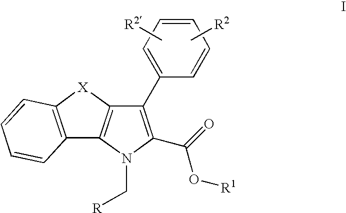
   in which
is N—R³, O or S,
R is 2,1,3-benzothiadiazol-4- or 5-yl or 2,1-benzoisothiazol-5-or 6-yl, each of which is unsubstituted or monosubstituted or disubstituted by R² and/or R^{2′}, or
   phenyl which is unsubstituted or monosubstituted, disubstituted or trisubstituted by R² and/or R^{2′},
R¹ is H or A,
R² and R^{2′} are each, independently of one another, H, A, OH, OA, Hal, OCF₃, OCHF₂, —O—CO-A, —O-alkylene-COOR¹, —O-alkylene-CH₂—OR¹,
   or
   OCH₂-phenyl or —O—CO-phenyl, each of which is unsubstituted or monosubstituted or disubstituted in the phenyl moiety by R⁴ and/or R^{4′},
R² and R^{2′} together are alternatively —OCH₂O—, —OCH₂CH₂O— or —OCH₂CH₂—,
R³ is H, A, alkylene-O-A, —CO—OA, or alkylene-phenyl which is unsubstituted or monosubstituted or disubstituted in the phenyl moiety by R⁴ and/or R^{4′},
R⁴ and R^{4′} are each, independently of one another, H. A, OH, OA, Hal, COOR¹ or CH₂OR¹,
A is alkyl having 1-6 carbon atoms,
Hal is fluorine, chlorine, bromine or iodine,
and salts thereof;
q) the compounds of the formula I described in WO 9905132 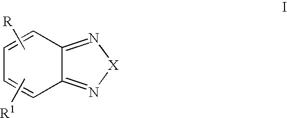
   in which
R is 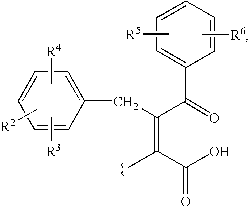
X is O or S,
R¹ is H, Hal, OA or A,
R², R³, R⁵ and R⁶ are each, independently of one another, H, Hal, A, OA or R⁴,
R⁴ is —O—(CH₂)ₙ—Cy,
Cy is cycloalkyl having 3-8 carbon atoms,
A is alkyl having 1-6 carbon atoms, in which one or two CH₂ groups may be replaced by O or S atoms or by —CR⁵═CR^{5′}— groups and/or 1-7 H atoms may be replaced by F,
R⁵ and R^{5′} are each, independently of one another, H, F or A,
Hal is fluorine, chlorine, bromine or iodine,
n is 0, 1 or 2,
or a tautomeric cyclised form, and the (E)-isomers and the salts of all isomers,
   for the preparation of a medicament for inhibiting the growth of neoplastic cells.

The use of other endothelin receptor antagonists for the treatment of tumours is described, for example, in WO 99/06397, WO 98/57933 and WO 96/06095.

The invention had the object of providing novel uses of medicaments in the form of pharmaceutical preparations which have better properties than known medicaments which can be used for the same purposes.

Surprisingly, it has been found that the compounds of the formulae I described above are suitable for the treatment of cancer diseases.

The compounds of the formula I described above and salts thereof exhibit very valuable pharmacological properties and are well tolerated. The compounds exhibit, inter alia, high affinity to the endothelin sub-receptors ET_{A} and ET_{B}. These actions can be determined by conventional in-vitro or in-vivo methods, as described, for example, by P. D. Stein et al., J. Med. Chem. 37, 1994, 329-331 and E. Ohlstein et al., Proc. Natl. Acad. Sci. U.S.A 91, 1994, 8052-8056.

The compounds of the formula I can be employed as medicament active ingredients in human and veterinary medicine. They can furthermore be employed as intermediates for the preparation of further medicament active ingredients.

The term “neoplastic cells” is taken to mean cancer cells.

Endothelin plays a role in the following types of cancer:

Prostate Cancer:

Prostate cancer cells secrete endothelin 1, patients with prostate cancer undergoing metastasis have a higher ET-1 plasma level, ET 1 stimulates proliferation of various prostate cancer cell lines, ET-1 stimulates osteo-blasts (Nelson J B et al. Nature Medicine 1/9 944-949, 1995).

ET-1 stimulates bone formation in an osteoblast tumour model, ET-1 influences metastasis formation by prostate cancer (Nelson JB et al., Urology 53/5, 1064-1069, 1999).

Atrasentan (Abbott, endothelin A receptor antagonist) inhibits the growth of various prostate cancer cell lines in vitro (Nelson J B et al. Cancer Research 56, 663-668, 1996).

Ovarian Carcinoma:

Expression of endothelin 1 and endothelin A receptor (ETAR) in ovarian carcinomas, ET-1 stimulates proliferation of primary ovarian carcinoma cells, BQ123 (selective endothelin A receptor antagonist) inhibits the proliferation of tumour cells (Bag nato A et al. Cancer Res 59, 720-727, 1999). Expression of ET1 and ETAR in ovarian carcinomas (Salani D et al. American Journal of Pathology 157/5, 1537-1547, 2000).

ET-1 protects ovarian carcinoma cells against apoptosis. This can be eliminated by BQ123 (selective endothelin A receptor antagonist) (Del Bufalo D et al., Molecular Pharmacology 61/3, 524532, 2002).

Intestinal Cancer:

Overexpression of ETAR in intestinal tumours (Ali H et al., Journal of Cardiovascular Pharmacology 36 S1 S69-S71, 2000).

ET-1 stimulates the proliferation of intestinal cancer cell lines. This can be inhibited by BQ123 and BQ610 (selective endothelin Areceptor antago-nists) (Ali H et al. Gut 47, 685-688, 2000).

ET-1 is overexpressed in tumours in intestinal cancer patients. BQ123 (selective endothelin A receptor antagonist) inhibits metastasis formation in a rat metastasis model (Asham E et al. British Journal of Cancer 81/11, 1759-1763, 2001).

Cervical Carcinoma:

HPV positive cervical carcinomas express ET-1 and overexpress endothelin A receptor. ET-1 stimulates proliferation of tumour cells. This can be inhibited by BQ123 (Venuti A et al., FASEB 14/14, 2279-2283, 2000).

Melanoma:

In melanomas, the endothelin B receptor is more important:

Melanoma cells overexpress endothelin B receptor.

Bosetan, an endothelin A and endothelin B receptor antagonist, inhibits the proliferation of melanoma cells in vitro (AACR Abstract No. 358, 2002).

Pancreas:

Ro 61-612/001, an endothelin A and endothelin B receptor antagonist, inhibits the proliferation of pancreas tumour cells (ASPC-1) in vivo (AACR Abstract No. 3365, 2000, no paper published to date).

In-Vivo Experiment:

Testing of the substance in an ovarian carcinoma cell line analogously to AACR Abstract No. 2075, 2000: Rosano L et al., Inhibition of tumour growth and angiogenesis by ABT 627 an endothelin receptor A antagonist in ovarian carcinoma xenografts.

The effect of endothelin receptor antagonists in the treatment of cancer can also be determined by the method described by Shichiri et al. in J. Clin. Invest. 87, 1867 (1991).

The invention preferably relates to the use of endothelin receptor antagonists selected from the group consisting of
i) the compounds described in EP 0733626
   a) 5-bromo-2-ethyl-N-(2,1,3-benzothiadiazol-5-yl)benzenesulfonamide;
   b) 2,5-dichloro-N-(2,1,3-benzothiadiazol-5-yl)benzenesulfonamide;
   c) 5-bromo-2-propyl-N-(2,1,3-benzothiadiazol-5-yl)benzenesulfonamide;
   d) 5-dimethylamino-N-(2,1,3-benzothiadiazol-5-yl)naphthalenesulfonamide;
   e) 5-dimethylamino-N-[6-methyl-(2,1,3-benzothiadiazol-5-yl)]-naphthalenesulfonamide;
   f) 5-dimethylamino-N-[4-bromo-(2,1,3-benzothiadiazol-5-yl)]-naphthalenesulfonamide;
   g) 5-dimethylamino-N-(2,1,3-benzothiadiazol-4-yl)naphthalenesulfonamide;
   h) 5-dimethylamino-N-([1,2,5]-oxadiazole-[3,4-b]-pyridin-6-yl)naphthalenesulfonamide;
   i) 5-dimethylamino-N-(1,2,5-benzoxadiazol-5-yl)-1-naphthalenesulfonamide;
   j) 5-dimethylamino-N-(6-bromo-7-methyl-1,2,5-benzoxadiazol-5-yl)-1-naphthalenesulfonamide;
   k) 2-phenyl-N-(2,1,3-benzothiadiazol-5-yl)benzenesulfonamide;
ii) the compounds described in EP 0758650
   a) 2-(1,3-benzodioxol-5-yl)-2-(1,3-dihydro-1,3-dioxoisoindol-5-yloxy)acetic acid;
   b) 2-(1,3-benzodioxol-5-yl)-2-(1,3-dihydro-1,3-dioxoisoindol-5-yloxy)-N-(4-tert-butylphenylsulfonyl)acetamide;
   c) 2-(1,3-benzodioxol-5-yl)-2-(1,3-dihydro-1,3-dioxoisoindol-5-yloxy)-N-(4-isopropylphenylsulfonyl)acetamide;
   d) 2-(1,3-benzodioxol-5-yl)-2-(7-propylquinolin-8-yloxy)acetic acid;
   e) 2-(1,3-benzodioxol-5-yl)-2-(7-propylquinolin-8-yloxy)-N-(4-tert-butylphenylsulfonyl)acetamide;
   f) 2-(1,3-benzodioxol-5-yl)-2-(6-propylindol-7-yloxy)acetic acid;
   g) 2-(1,3-benzodioxol-5-yl)-2-(1-methyl-2-propylbenzimidazol-4-yloxy)acetic acid;
iii) the compounds described in EP 0755934
   a) 1,2-dihydro-1-(2-methoxybenzyl)-4-(4-methoxyphenyl)-2-oxo-benzofuro[3,2-b]pyridine-3-carboxylic acid;
   b) 2-(2-methoxybenzyloxy)-4-(4-methoxyphenyl)benzofuro[3,2-b]-pyridine-3-carboxylic acid;
   c) 4-(1,4-benzodioxan-6-yl)-1,2-dihydro-1-(2-methoxybenzyl)-2-oxobenzofuro[3,2-b]pyridine-3-carboxylic acid;
   d) 2-(2-methoxyphenoxy)-4-(4-methoxyphenyl)benzofuro[3,2-b]-pyridine-3-carboxylic acid;
   e) 4-(1,4-benzodioxan-6-yl)-1,2-dihydro-1-(2-methoxybenzyl)-2-oxo-3-(1H-tetrazol-5-yl)benzofuro[3,2-b]pyridine;
   f) 1,2-dihydro-1-(2,3-methylenedioxybenzyl)-4-(4-methoxyphenyl)-2-oxobenzofuro[3,2-b]pyridine-3-carboxylic acid;
   g) 1,2-dihydro-1-(2,3-methylenedioxybenzyl)-7-methyl-4-(4-tri-fluoromethoxyphenyl)-2-oxobenzofuro[3,2-b]pyridine-3-carboxylic acid;
   h) 1,2-dihydro-1-(2,3-methylenedioxybenzyl)-7-methyl-4-(4-methoxyphenyl)-2-oxobenzothieno[3,2-b]pyridine-3-carboxylic acid;
   i) 1,2-dihydro-1-(2,1,3-benzothiadiazol-5-methyl)-4-(4-methoxyphenyl)-2-oxobenzofuro[3,2-b]pyridine-3-carboxylic acid;
iv) the compounds described in EP 0757039
   a) 4-(1,3-benzodioxol-5-yl)-1,2-dihydro-1-(2-methoxybenzyl)-2-oxoquinoline-3-carboxylic acid;
   b) 4-(1,3-benzodioxol-5-yl)-1,2-dihydro-1-(4-methoxybenzyl)-2-oxoquinoline-3-carboxylic acid;
   c) 4-(1,3-benzodioxol-5-yl)-1,2-dihydro-1-(3,4-methylenedioxybenzyl)-2-oxoquinoline-3-carboxylic acid;
   d) 4-(1,3-benzodioxol-5-yl)-1,2-dihydro-1-(2-methoxybenzyi)-2-oxoquinoline-3-acetic acid;
   e) 4-(1,3-benzodioxol-5-yl)-1,2-dihydro-1-(3,4-methylenedioxybenzyl)-2-oxoquinoline-3-acetic acid;
   f) 4-(1,3-benzodioxol-5-yl)-1,2-dihydro-6-ethoxy-1-(2-methoxybenzyl)-2-oxoquinoline-3-carboxylic acid;
   g) 4-(1,3-benzodioxol-5-yl)-1,2-dihydro-6-ethoxy-1-(4-methoxybenzyl)-2-oxoquinoline-3-carboxylic acid;
   h) 4-(1,3-benzodioxol-5-yl)-1,2-dihydro-6-ethoxy-1-(6-chloro-3,4-methylenedioxybenzyl)-2-oxoquinoline-3-carboxylic acid;
   i) 4-(1,3-benzodioxol-5-yl)-1,2-dihydro-6-ethoxy-1-(3,4-methylenedioxybenzyl)-2-oxoquinoline-3-carboxylic acid;
   j) 4-(1,3-benzodioxol-5-yl)-1,2-dihydro-6-ethoxy-1-(3-methoxybenzyl)-2-oxoquinoline-3-carboxylic acid;
v) the compounds described in EP 0796250
   a) 2-(1,3-benzodioxol-5-yl)-2-(2,3-dihydro-4,6-dimethylpyridazin-3-on-2-yl)-N-(4-isopropylphenylsulfonyl)acetamide;
   b) 2-(1,3-benzodioxol-5-yl)-2-(6-(4-methoxyphenyl)-2,3,4,5-tetra-hydropyridazin-3-on-2-yl)-N-(4-isopropylphenylsulfonyl)-acetamide;
   c) 2-(1,3-benzodioxol-5-yl )-2-(6-(4-chlorophenyl)-2,3,4,5-tetrahydropyridazin-3-on-2-yl)-N-(4-isopropylphenylsulfonyl)-acetamide;
   d) 2-(1,3-benzodioxol-5-yl)-2-(6-(3,4-dimethoxyphenyl)-2,3,4,5-tetrahydropyridazin-3-on-2-yl)-N-(4-isopropylphenylsulfonyl)-acetamide;
   e) 2-(1,3-benzodioxol-5-yl)-2-(4-methyl-6-phenyl-2,3-dihydro-pyridazin-3-on-2-yl)-N-(4-isopropylphenylsulfonyl)acetamide;
   f) 2-(1,3-benzodioxoi-5-yl)-2-(5-(3,4-dimethoxyphenyl)-6-ethyl-2H-3,6-dihydro-1,3,4-thiadiazin-2-on-3-yl)-N-(4-isopropylphenyl-sulfonyl)acetamide;
vi) the compounds described in WO 9719077
   a) 3-(1,3-benzodioxol-5-yl)-1-(2,1,3-benzothiadiazol-5-ylmethyl)-5-propoxyindole-2-carboxylic acid;
   b) 3-(4-methoxyphenyl)-1-(2,1,3-benzothiadiazol-5-ylmethyl)-5-ethoxyindole-2-carboxylic acid;
   c) 3-(4-methoxyphenyl)-1-(2,1,3-benzothiadiazol-5-ylmethyl)-5-propoxyindole-2-carboxylic acid;
   d) 3-(2,1,3-benzothiadiazol-5-yl)-1-(4-methoxybenzyl)-5-ethoxyindole-2-carboxylic acid;
   e) 3-(2,1,3-benzothiadiazol-5-yl)-1-(4-methoxybenzyl)-5-propoxyindole-2-carboxylic acid;
   f) 3-(2,1,3-benzothiadiazol-5-yl)-1-(3,4-methylenedioxybenzyl)-5,6-dimethoxyindole-2-carboxylic acid;
vii) the compounds described in WO 9730982
   2-(2,1,3-benzothiadiazol-5-yl)-3-benzyl4-(4-methoxyphenyl)-4-oxo-2-butenoic acid;
   2-(2,1,3-benzothiadiazol-5-yl)-3-(3,4,5-trimethoxybenzyl)-4-(4-methoxyphenyl)-4-oxo-2-butenoic acid;
   2-(2,1,3-benzothiadiazol-5-yl)-3-(3,4-diisopropoxy-5-methoxybenzyl)-4-(4-methoxyphenyl)-4-oxo-2-butenoic acid;
   2-(2,1,3-benzothiadiazol-5-yl)-3-benzyl-4-(1,4-benzodioxan-6-yl)-4-oxo-2-butenoic acid;
   2-(2,1,3-benzothiadiazol-5-yl)-3-(3,4,5-trimethoxybenzyl)-4-(1,4-benzodioxan-6-yl)-4-oxo-2-butenoic acid;
   2-(2,1,3-benzothiadiazol-5-yl)-3-(3,4-diisopropoxy-5-methoxybenzyl)-4-(1,4-benzodioxan-6-yl)-4-oxo-2-butenoic acid;
   2-(2,1,3-benzothiadiazol-5-yl)-3-(3,4,5-trimethoxybenzyl)-4-(1,3-benzodioxol-5-yl)-4-oxo-2-butenoic acid;
   3-(2,1,3-benzothiadiazol-5-yl)-4-benzyl-5-hydroxy-5-(3-fluoro-4-methoxyphenyl)-5H-furan-2-one;
   2-(2,1,3-benzothiadiazol-5-yl)-3-(3,4,5-trimethoxybenzyl)-4-(3-fluoro-4-methoxyphenyl)-4-oxo-2-butenoic acid;
   3-(2,1,3-benzothiadiazol-5-yl)-4-[(7-methoxy-1,3-benzodioxol-5-yl)methyl]-5-hydroxy-5-(4-methoxyphenyl)-5H-furan-2-one;
   3-(2,1,3-benzothiadiazol-5-yl)-4-(3,4,5-trimethoxybenzyl)-5-hydroxy-5-(3-methoxyphenyl)-5H-furan-2-one;
   3-(2,1,3-benzothiadiazol-5-yl)-4-(3,4,5-trimethoxybenzyl)-5-hydroxy-5-(2-methoxyphenyl)-5H-furan-2-one;
   3-(2,1,3-benzothiadiazol-5-yl)-4-(4-methylthiobenzyl)-5-hydroxy-5-(4-methoxyphenyl)-5H-furan-2-one;
   3-(2,1,3-benzothiadiazol-5-yl)-4-(3-benzyloxy-4-methoxybenzyl)-5-hydroxy-5-(4-methoxyphenyl)-5H-furan-2-one;
   3-(2,1,3-benzothiadiazol-5-yl)-4-(2,3-dihydrobenzofuran-5-yl-methyl)-5-hydroxy-5-(4-methoxyphenyl)-5H-furan-2-one;
   3-(2,1,3-benzothiadiazol-5-yl)-4-(2-methylpropyl)-5-hydroxy-5-(4-methoxyphenyl)-5H-furan-2-one;
   3-(2,1,3-benzothiadiazol-5-yl)-4-(3,5-dimethoxybenzyl)-5-hydroxy-5 -(4-methoxyphenyl)-5H-furan-2-one;
   3-(2,1,3-benzothiadiazol-5-yl)-4-(4-tert-butoxybenzyl)-5-hydroxy-5-(4-methoxyphenyl)-5H-furan-2-one;
   3-(2,1,3-benzothiadiazol-5-yl)-4-(4-hydroxybenzyl)-5-hydroxy-5-(4-methoxyphenyl)-5H-furan-2-one;
   3-(2,1,3-benzothiadiazol-5-yl)-4-(4-trifluoromethoxybenzyl)-5-hydroxy-5-(4-methoxyphenyl)-5H-furan-2-one;
   3-(2,1,3-benzothiadiazol-5-yl)-4-(3,5-dimethoxy-4-isopropoxybenzyl)-5-hydroxy-5-(4-methoxyphenyl)-5H-furan-2-one;
   3-(2,1,3-benzothiadiazol-5-yl)-4-(3,5-dimethoxy-4-pentyloxybenzyl)-5-hydroxy-5-(4-methoxyphenyl)-5H-furan-2-one;
   3-(2,1,3-benzothiadiazol-5-yl)-4-(3,5-dimethoxy4-hexyloxybenzyl)-5-hydroxy-5-(4-methoxyphenyl)-5H-furan-2-one;
   3-(2,1,3-benzothiadiazol-5-yl)-4-(4-phenoxybenzyl)-5-hydroxy-5-(4-methoxyphenyl)-5H-furan-2-one;
   3-(2,1,3-benzothiadiazol-5-yl)-4-(4,5-dimethoxy-3-isopropoxybenzyl)-5-hydroxy-5-(4-methoxyphenyl)-5H-furan-2-one;
   3-(2,1,3-benzothiadiazol-5-yl)-4-(3,4,5-trimethoxybenzyl)-5-hydroxy-5-(2,5-dimethoxyphenyl)-5H-furan-2-one;
   3-(2,1,3-benzothiadiazol-5-yl)-4-(3,4,5-trimethoxybenzyl)-5-hydroxy-5-(3-chloro-4-methoxyphenyl)-5H-furan-2-one;
   3-(2,1,3-benzothiadiazol-5-yl)-4-(3,4,5-trimethoxybenzyl)-5-hydroxy-5-(3-methyl-4-methoxyphenyl)-5H-furan-2-one;
   3-(2,1,3-benzothiadiazol-5-yl)-4-(3,4-diisopropoxy-5-methoxybenzyl)-5-hydroxy-5-(2,5-dimethoxyphenyl)-5H-furan-2-one;
   3-(2,1,3-benzothiadiazol-5-yl)-4-(3,4,5-trimethoxybenzyl)-5-hydroxy-5-(2,3-dihydrobenzofuran-5-yl)-5H-furan-2-one;
   3-(2,1,3-benzothiadiazol-5-yl)-4-(3,5-dimethoxy-4-isopropoxybenzyl)-5-hydroxy-5-(3-fluoro-4-methoxyphenyl)-5H-furan-2-one;
   3-(2,1,3-benzothiadiazol-5-yl)-4-(3,4-dimethoxy-5-propoxybenzyl)-5-hydroxy-5-(4-methoxyphenyl)-5H-furan-2-one;
   3-(2,1,3-benzothiadiazol-5-yl)-4-(3,4-diisopropoxy-5-methoxybenzyl)-5-hydroxy-5-(4-propoxyphenyl)-5H-furan-2-one;
   3-(2,1,3-benzothiadiazol-5-yl)-4-(3,4-diisopropoxy-5-methoxybenzyl)-5-hydroxy-5-(2,4-dimethoxyphenyl)-5H-furan-2-one;
   3-(2,1,3-benzothiadiazol-5-yl)-4-(4-benzyloxy-2-methoxybenzyl)-5-hydroxy-5-(4-methoxyphenyl)-5H-furan-2-one;
   3-(2,1,3-benzothiadiazol-5-yl)-4-(2,3,4-trimethoxybenzyl)-5-hydroxy-5-(4-methoxyphenyl)-5H-furan-2-one;
   3-(2,1,3-benzothiadiazol-5-yl)-4-(3,4,5-trimethoxybenzyl)-5-hydroxy-5-(2,4-dimethoxyphenyl)-5H-furan-2-one;
   3-(2,1,3-benzothiadiazol-5-yl)-4-(3,4,5-triethoxybenzyl)-5-hydroxy-5-(4-methoxyphenyl)-5H-furan-2-one;
   3-(2,1,3-benzothiadiazol-5-yl)-4-(3,4,5-trimethoxybenzyl)-5-hydroxy-5-(4-difluoromethoxyphenyl)-5H-furan-2-one;
   3-(2,1,3-benzothiadiazol-5-yl)-4-(3-hydroxy-4-methoxybenzyl)-5-hydroxy-5-(4-methoxyphenyl)-5H-furan-2-one;
   3-(2,1,3-benzothiadiazol-5-yl)-4-(2,4-dimethoxybenzyl)-5-hydroxy-5-(4-methoxyphenyl)-5H-furan-2-one;
   3-(2,1,3-benzothiadiazol-5-yl)-4-(2,4,5-trimethoxybenzyl)-5-hydroxy-5-(4-methoxyphenyl)-5H-furan-2-one;
   3-(2,1,3-benzothiadiazol-5-yl)-4-(3,4,5-trimethoxybenzyl)-5-hydroxy-5-(3-fluoro-4-isopropoxyphenyl)-5H-furan-2-one;
   3-(2,1,3-benzothiadiazol-5-yl)-4-(3,4,5-trimethoxybenzyl)-5-hydroxy-5-(3-fluoro-4-propoxyphenyl)-5H-furan-2-one;
   3-(2,1,3-benzothiadiazol-6-methyl-5-yl)-4-(3,5-dimethoxy-4-isopropoxybenzyl)-5-hydroxy-5-(4-methoxyphenyl)-5H-furan-2-one;
   3-(2,1,3-benzothiadiazol-5-yl)-4-(3,5-dimethoxy-4-benzyloxy-benzyl)-5-hydroxy-5-(4-methoxyphenyl)-5H-furan-2-one;
   3-(2,1,3-benzothiadiazol-5-yl)-4-(3,5-dimethoxy-4-hydroxy-benzyl)-5-hydroxy-5-(4-methoxyphenyl)-5H-furan-2-one;
   3-(2,1,3-benzothiadiazol-5-yl)-4-(3,5-dimethoxy-4-propoxy-benzyl)-5-hydroxy-5-(4-methoxyphenyl)-5H-furan-2-one;
   3-(2, 1,3-benzothiadiazol-5-yl)-4-(3,4-dimethoxy-5-isopropoxybenzyl)-5-hydroxy-5-(1,4-benzodioxan-6-yl)-5H-furan-2-one;
   3-(2,1,3-benzothiadiazol-6-methyl-5-yl)-4-(3,4,5-trimethoxybenzyl)-5-hydroxy-5-(4-methoxyphenyl)-5H-furan-2-one;
   3-(2,1,3-benzothiadiazol-5-yl)-4-(4-isopropoxybenzyl)-5-hydroxy-5-(4-methoxyphenyl)-5H-furan-2-one;
   3-(2,1,3-benzothiadiazol-5-yl)-4-(4-hexyloxybenzyl )-5-hydroxy-5-(4-methoxyphenyl)-5H-furan-2-one;
   3-(2,1,3-benzothiadiazol-5-yl)-4-(3,5-dimethoxy-4-isopropoxybenzyl)-5-hydroxy-5-(1,4-benzodioxan-6-yl)-5H-furan-2-one;
   3-(2,1,3-benzothiadiazol-5-yl)-4-(3-methoxy-5-butoxybenzyl)-5-hydroxy-5-(4-methoxyphenyl)-5H-furan-2-one;
   3-(2,1,3-benzothiadiazol-5-yl)-4-(3,4-diisopropoxybenzyl)-5-hydroxy-5-(4-methoxyphenyl)-5H-furan-2-one;
   3-(2,1,3-benzothiadiazol-5-yl)-4-(3,4,5-trimethoxybenzyl)-5-hydroxy-5-(2-fluoro-4-methoxyphenyl)-5H-furan-2-one;
   3-(2,1,3-benzothiadiazol-5-yl)-4-(3,4-dimethoxy-5-isopropoxybenzyl)-5-hydroxy-5-(3-fluoro-4-methoxyphenyl)-5H-furan-2-one;
   3-(2,1,3-benzothiadiazol-5-yl)-4-(3,4-dimethoxy-5-benzyloxy-benzyl)-5-hydroxy-5-(4-methoxyphenyl)-5H-furan-2-one;
   3-(2,1,3-benzothiadiazol-5-yl)-4-(3,4,5-trimethoxybenzyl)-5-hydroxy-5-(4-fluoro-2-methoxyphenyl)-5H-furan-2-one;
   3-(2,1,3-benzothiadiazol-5-yl)-4-(3,5-dimethoxy-5-ethoxybenzyl)-5-hydroxy-5-(4-methoxyphenyl)-5H-furan-2-one;
   3-(2,1,3-benzothiadiazol-5-yl)-4-(4-methoxycarbonylbenzyl)-5-hydroxy-5-(4-methoxyphenyl)-5H-furan-2-one;
   3-(2,1,3-benzothiadiazol-5-yl)-4-(3,4-diisopropoxybenzyl)-5-hydroxy-5-(3-fluoro-4-methoxyphenyl)-5H-furan-2-one;
   3-(2,1,3,-benzothiadiazol-5-yl)-4-(3,4,5-trimethoxybenzyl)-5-hydroxy-5-(4-benzyloxyphenyl)-5H-furan-2-one;
   3-(2,1,3,-benzothiadiazol-4-methyl-5-yl)-4-(3,4,5-trimethoxybenzyl)-5-hydroxy-5-(4-methoxyphenyl)-5H-furan-2-one;
   3-(2,1,3-benzothiadiazol-5-yl)-4-(3,5-dimethoxy-4-isobutoxybenzyl)-5-hydroxy-5-(4-methoxyphenyl)-5H-furna-2-one;
   4-(2,1,3-benzothiadiazol-5-ylmethyl)-3-(7-methoxy-1,3-benzo-dioxol-5-yl)-5-hydroxy-5-(4-methoxyphenyl)-5H-furan-2-one;
   2-(2,1,3-benzothiadiazol-5-yl)-3-(3,4-diisopropoxy-5-methoxybenzyl)-4-(3-fluoro-4-methoxyphenyl)-4-oxo-2-butenoic acid;
   2-(2,1,3-benzothiadiazol-5-yl)-3-(3,5-dimethoxy-4-isopropoxybenzyl)-4-(4-methoxyphenyl)-4-oxo-2-butenoic acid;
   2-(2,1,3-benzothiadiazol-5-yl)-3-(3,4-dimethoxy-5-isopropoxybenzyl)-4-(4-methoxyphenyl)-4-oxo-2-butenoic acid;
   2-(2,1,3-benzothiadiazol-5-yl)-3-(3,5-dimethoxy4-isopropoxybenzyl)-4-(3-fluoro-4-methoxyphenyl)-4-oxo-2-butenoic acid;
   2-(2,1,3-benzothiadiazol-5-yl)-3-(3,4,5-trimethoxybenzyl)-4-(⁴-methoxyphenyl)-4-oxo-2-butenoic acid;
viii) the compounds described in WO 9730996
   a) 3-(2,1,3-benzothiadiazol-5-aminosulfonyl)-N-(6-methyl-1,3-benzodioxol-5-yl)thiophene-2-carboxamide;
   b) 3-(2,1,3-benzothiadiazol-5-aminosuifonyl)-N-(6-acetyl-1,3-benzodioxol-5-yl)thiophene-2-carboxamide;
   c) 3-(2,1,3-benzothiadiazol-5-aminosulfonyl)-N-(6-cyano-1,3-benzodioxol-5-yl)thiophene-2-carboxamide;
   d) 3-(2,1,3-benzothiadiazol-5-aminosulfonyl)-2-(6-methyl-1,3-benzodioxol-5-ylmethylcarbonyl)thiophene;
ix) the compounds described in DE 19609597
   a) N-(2,1,3-benzothiadiazol-5-yl)-5-N′-isopropylamino-1-naphthalenesulfonamide;
   b) N-(2,1,3-benzothiadiazol-5-yl)-5-N′-propylamino-1-naphthalenesulfonamide;
   c) N-(2,1,3-benzothiadiazol-5-yl)-5-N′-methylamino-1-naphthalenesulfonamide;
   d) N-(2,1,3-benzothiadiazol-5-yl)-5-N′-ethylamino-1-naphthalenesulfonamide;
   e) N-(2,1,3-benzothiadiazol-5-yl)-5-N′-butylamino-1-naphthalenesulfonamide;
x) the compounds described in DE 19612101
   a) 4-(4-methoxyphenyl)-1,6-dihydro-1-(2-methoxybenzyl)-2-methyl-6-oxopyrimidine-5-carboxylic acid;
   b) 4-(3,4-methylenedioxyphenyl)-1,6-dihydro-1-(2-methoxybenzyl)-2-cyclopropyl-6-oxopyrimidine-5-carboxylic acid;
   c) 4-(2-carboxy4-methoxy-7-benzofurnayl)-1,6-dihydro-1-(2-methoxybenzyl)-2-methyl-6-oxopyrimidine-5-carboxylic acid;
   d) 4-(2-phenyl-4-methoxyphenyl)-1,6-dihydro-1-(2-methoxybenzyl)-2-methyl-6-oxopyrimidine-5-carboxylic acid;
   e) 4-(2-carboxy-4-methoxy-7-benzofuranyl)-1,6-dihydro-1-(5-benzothiadiazolyl)-2-methyl-6-oxopyrimidine-5-carboxylic acid;
   f) 4-(4-methoxyphenyl)-1,6-dihydro-1-(5-benzothiadiazolyl)-2-methyl-6-oxopyrimidine-5-carboxylic acid;
xi) the compounds described in WO 9827091
   a) 4-(2,1,3-benzothiadiazol-5-ylmethyl)-1-benzyl-3-butyl-1H-pyrazole-5-carboxylic acid;
   b) 4-(2,1,3-benzothiadiazol-5-ylmethyl)-1-(3-methoxybenzyl)-3-butyl-1H-pyrazole-5-carboxylic acid;
   c) 4-(2,1,3-benzothiadiazol-6-chloro-5-ylmethyl)-1-(3-methoxybenzyl)-3 -butyl-1H-pyrazole-5-carboxylic acid;
   d) 4-(2,1,3-benzothiadiazol-5-ylmethyl)-1-(2-carboxymethoxy-4-methoxybenzyl)-3-butyl-1H-pyrazole-5-carboxylic acid;
   e) 4-(2,1,3-benzothiadiazol-5-ylmethyl)-1-(2,4-dimethoxybenzyl)-3-butyl-1H-pyrazole-5-carboxylic acid;
   f) 4-(2,1,3-benzothiadiazol-5-ylmethyl)-1-(3-methoxybenzyl)-3-phenyl-1H-pyrazole-5-carboxylic acid;
   g) 4-(2,1,3-benzothiadiazol-5-ylmethyl)-1-(3-methoxybenzyl)-3-(2-thienyl)-1H-pyrazole-5-carboxylic acid;
   h) 4-(2,1,3-benzothiadiazol-5-ylmethyl)-1-(3-methoxybenzyl)-3-cyclohexyl-1H-pyrazole-5-carboxylic acid;
   i) 4-(2,1,3-benzothiadiazol-5-ylmethyl)-1-(2-carboxymethoxy4-methoxybenzyl)-3-propoxy-1H-pyrazole-5-carboxylic acid;
xii) the compounds described in WO 9827077
   a) 2-(2,1,3-benzothiadiazol-5-yl)-3-(thien-2-ylmethyl)-4-(4-methoxyphenyl)-4-oxo-2-butenoic acid;
   b) 2-(2,1,3-benzothiadiazol-5-yl)-3-(5-methoxythien-2-ylmethyl)-4-(4-methoxyphenyl)-4-oxo-2-butenoic acid;
   c) 3-(2,1,3-benzothiadiazol-5-yl)-4-(furan-2-ylmethyl)-5-hydroxy-5-(4-methoxyphenyl)-5H-furan-2-one;.
   d) 3-(2,1,3-benzothiadiazol-5-yl)-4-(3,4,5-trimethoxybenzyl)-5-hydroxy-5-(3,4-dihydro-2H-1,5-benzodioxepin-7-yl)-5H-furan-2-one;
   e) 3-(2,1,3-benzothiadiazol-5-yl)-4-(3,4-diisopropoxy-5-methoxybenzyl)-5-hydroxy-5-(3,4-dihydro-2H-1,5-benzodioxepin-7-yl)-5H-furan-2-one;
   f) 3-(2,1,3-benzothiadiazol-5-yl)-4-(thien-3-ylmethyl)-5-hydroxy-5-(4-methoxyphenyl)-5H-furan-2-one;
xiii) the compounds described in WO 9841515
   a) 2-(2,1,3-benzothiadiazol-5-yl)-4-(4-methoxyphenyl)-4-oxo-2-butanoic acid;
   b) 2-(2,1,3-benzothiadiazol-5-yl)-3-(2,1,3-benzothiadiazol-5-yl-methyl)acetic acid;
   c) 2-(2,1,3-benzothiadiazol-5-yl)-2-(4-methoxycarbonylbenzyl)-acetic acid;
   d) 2-(2,1,3-benzothiadiazol-5-yl)-2-(4-methoxycarbonylbenzyl)-N-(4-isopropylphenyisulfonyl)acetamide;
   e) 2-(2,1,3-benzothiadiazol-5-yl)-2-(4-carboxybenzyl)-N-(4-isopropylphenylsulfonyl)acetamide;
   f) 2-(2,1,3-benzothiadiazol-5-yl)-4-(4-methoxybenzyl)acetic acid;
   g) 2-(2,1,3-benzothiadiazol-5-yl)-2-(4-methoxybenzyl)-4-(4-methoxyphenyl)-4-oxo-2-butanoic acid;
xiv) the compounds described in WO 9841521
   a) 2-(1,3-benzodioxol-5-yl)-3-(2,1,3-benzothiadiazol-5-yl)-succinic acid;
   b) 2,3-bis(1,3-benzodioxol-5-yl)maleic acid;
   c) N,N-dibutyl-2,3-bis(1,3-benzodioxol-5-yl)maleamide;
   d) 2,3-bis(1,3-benzodioxoi-5-yl)maleic anhydride;.
   e) 2-(1,3-benzodioxol-5-yl)-3-phenylmaleic anhydride;
xv) the compounds described in WO 9842702
   ethyl [3-(2,1,3-benzothiadiazol-5-yl)-4-(3,4,5-trimethoxybenzyl)-5-(4-methoxyphenyl)-5H-furan-2-on-5-yloxycarbonylamino]-acetate;
   ethyl [3-(2,1,3-benzothiadiazol-5-yl)-4-(3,4,5-trimethoxybenzyl)-5-(3-fluoro4-methoxyphenyl)-5H-furan-2-on-5-yloxycarbonyl-amino]acetate;
   N-1-naphthylethyl-[3-(2,1,3-benzothiadiazol-5-yl)-4-(3,4,5-trimethoxybenzyl)-5-(3-fluoro-4-methoxyphenyl)-5H-furna-2-on-5-yl]carbamate;
   ethyl 2-[3-(2,1,3-benzothiadiazol-5-yl)-4-(3,4,5-trimethoxybenzyl)-5-(3-fluoro-4-methoxyphenyl)-5H-furan-2-on-5-yloxycarbonyl-amino]-3-methylbutyrate;
   2-(2,1,3-benzothiadiazol-5-yl)-3-(3-fluoro-4-methoxybenzoyl)-4-(3,4,5-trimethoxyphenyl)but-2-enoic acid;
   3-(2,1,3-benzothiadiazol-5-yl)-4-benzyl-5-hydroxy-5-(3-fluoro-4-methoxyphenyl)-5H-furan-2-one;
   3-(2,1,3-benzothiadiazol-5-yl)-4-(3,4,5-trimethoxybenzyl)-5-hydroxy-5-(3-fluoro-4-methoxyphenyl)-5H-furna-2-one;
   3-(2,1,3-benzothiadiazol-5-yl)-4-[(7-methoxy-1,3-benzodioxol-5-yl)methyl]-5-hydroxy-5-(4-methoxyphenyl)-5H-furan-2-one;
   3-(2,1,3-benzothiadiazol-5-yl)-4-(3,4,5-trimethoxybenzyl)-5-hydroxy-5-(3-methoxyphenyl)-5H-furan-2-one;
   3-(2,1,3-benzothiadiazol-5-yl)-4-(3,4,5-trimethoxybenzyl)-5-hydroxy-5-(2-methoxyphenyl)-5H-furna-2-one;
   3-(2,1,3-benzothiadiazol-5-yl)-4-(4-methylthiobenzyl)-5-hydroxy-5-(4-methoxyphenyl)-5H-furan-2-one;
   3-(2,1,3-benzothiadiazol-5-yl)-4-(3-benzyloxy-4-methoxybenzyl)-5-hydroxy-5-(4-methoxyphenyl)-5H-furan-2-one;
   3-(2,1,3-benzothiadiazol-5-yl)-4-(2,3-dihydrobenzofuran-5-yl-methyl)-5-hydroxy-5-(4-methoxyphenyl)-5H-furan-2-one;
   3-(2,1,3-benzothiadiazol-5-yl)-4-(2-methylpropyl)-5-hydroxy-5-(4-methoxyphenyl)-5H-furan-2-one;
   3-(2,1,3-benzothiadiazol-5-yl)-4-(3,5-dimethoxybenzyl)-5-hydroxy-5-(4-methoxyphenyl)-5H-furan-2-one;
   3-(2,1,3-benzothiadiazol-5-yl)-4-(4-tert-butoxybenzyl)-5-hydroxy-5-(4-methoxyphenyl)-5H-furan-2-one;
   3-(2,1,3-benzothiadiazol-5-yl)-4-(4-hydroxybenzyl)-5-hydroxy-5-(4-methoxyphenyl)-5H-furna-2-one;
   3-(2,1,3-benzothiadiazol-5-yl)-4-(4-trifluoromethoxybenzyl)-5-hydroxy-5-(4-methoxyphenyl)-5H-furan-2-one;
   3-(2,1,3-benzothiadiazol-5-yl)-4-(3,5-dimethoxy-4-isopropoxy-benzyl)-5-hydroxy-5-(4-methoxyphenyl)-5H-furan-2-one;

3-(2,1,3-benzothiadiazol-5-yl)-4-(3,5-dimethoxy-4-pentyloxybenzyl)-5-hydroxy-5-(4-methoxyphenyl)-5H-furan-2-one;
3-(2,1,3-benzothiadiazol-5-yl )-4-(3,5-dimethoxy-4-hexyloxybenzyl)-5-hydroxy-5-(4-methoxyphenyl)-5H-furan-2-one;
3-(2,1,3-benzothiadiazol-5-yl)-4-(4-phenoxybenzyl)-5-hydroxy-5-(4-methoxyphenyl)-5H-furan-2-one;
3-(2,1,3-benzothiadiazol-5-yl)-4-(4,5-dimethoxy-3-isopropoxybenzyl)-5-hydroxy-5-(4-methoxyphenyl )-5H-furna-2-one;
3-(2,1,3-benzothiadiazol-5-yl)-4-(3,4,5-trimethoxybenzyl)-5-hydroxy-5-(2,5-dimethoxyphenyl)-5H-furan-2-one;
3-(2,1,3-benzothiadiazol-5-yl)-4-(3,4,5-trimethoxybenzyl)-5-hydroxy-5-(3-chloro-4-methoxyphenyl)-5H-furan-2-one;
3-(2,1,3-benzothiadiazol-5-yl)-4-(3,4,5-trimethoxybenzyl)-5-hydroxy-5-(3-methyl-4-methoxyphenyl)-5H-furan-2-one;
3-(2,1,3-benzothiadiazol-5-yl)-4-(3,4-diisopropoxy-5-methoxybenzyl)-5-hydroxy-5-(2,5-dimethoxyphenyl)-5H-furan-2-one;
3-(2,1,3-benzothiadiazol-5-yl)-4-(3,4,5-trimethoxybenzyl)-5-hydroxy-5-(2,3-dihydrobenzofuran-5-yl)-5H-furan-2-one;
3-(2,1,3-benzothiadiazol-5-yl)-4-(3,5-dimethoxy4-isopropoxybenzyl)-5-hydroxy-5-(3-fluoro-4-methoxyphenyl)-5H-furan-2-one;
3-(2,1,3-benzothiadiazol-5-yl)-4-(3,4-dimethoxy-5-propoxybenzyl)-5-hydroxy-5-(4-methoxyphenyl)-5H-furan-2-one;
   3-(2,1,3-benzothiadiazol-5-yl)-4-(3,4-diisopropoxy-5-methoxybenzyl)-5-hydroxy-5-(4-propoxyphenyl)-5H-furan-2-one;
   3-(2,1,3-benzothiadiazol-5-yl)-4-(3,4-diisopropoxy-5-methoxybenzyl)-5-hydroxy-5-(2,4-dimethoxyphenyl)-5H-furan-2-one;
   3-(2,1,3-benzothiadiazol-5-yl)-4-(4-benzyloxy-2-methoxybenzyl)-5-hydroxy-5-(4-methoxyphenyl)-5H-furan-2-one;
   3-(2,1,3-benzothiadiazol-5-yl)-4-(2,3,4-trimethoxybenzyl)-5-hydroxy-5-(4-methoxyphenyl)-5H-furan-2-one;
   3-(2,1,3-benzothiadiazol-5-yl)-4-(3,4,5-trimethoxybenzyl)-5-hydroxy-5-(2,4-d imethoxyphenyl)-5H-furna-2-one;
   3-(2,1,3-benzothiadiazol-5-yl)-4-(3,4,5-triethoxybenzyl)-5-hydroxy-5-(4-methoxyphenyl)-5H-furan-2-one;
   3-(2,1,3-benzothiadiazol-5-yl)-4-(3,4,5-trimethoxybenzyl)-5-hydroxy-5-(4-difluoromethoxyphenyl)-5H-furan-2-one;
   3-(2,1,3-benzothiadiazol-5-yl)-4-(3-hydroxy-4-methoxybenzyl)-5-hydroxy-5-(4-methoxyphenyl)-5H-furna-2-one;
   3-(2,1,3-benzothiadiazol-5-yl)-4-(2,4-dimethoxybenzyl)-5-hydroxy-5-(4-methoxyphenyl)-5H-furan-2-one;
   3-(2,1,3-benzothiadiazol-5-yl)-4-(2,4,5-trimethoxybenzyl)-5-hydroxy-5-(4-methoxyphenyl)-5H-furan-2-one;
   3-(2,1,3-benzothiadiazol-5-yl)-4-(3,4,5-trimethoxybenzyl)-5-hydroxy-5-(3-fluoro-4-isopropoxyphenyl)-5H-furan-2-one;
   3-(2,1,3-benzothiadiazol-5-yl)-4-(3,4,5-trimethoxybenzyl)-5-hydroxy-5-(3-fluoro-4-propoxyphenyl)-5H-furan-2-one;
   3-(2,1,3,-benzothiadiazol-6-methyl-5-yl)-4-(3,5-dimethoxy-4-isopropoxybenzyl)-5-hydroxy-5-(4-methoxyphenyl)-5H-furan-2-one;
   3-(2,1,3-benzothiadiazol-5-yl)-4-(3,5-dimethoxy4-benzyloxybenzyl)-5-hydroxy-5-(4-methoxyphenyl)-5H-furan-2-one;
   3-(2,1,3-benzothiadiazol-5-yl)-4-(3,5-dimethoxy-4-hydroxybenzyl)-5-hydroxy-5-(4-methoxypheriyl)-5H-furan-2-one;
   3-(2,1,3-benzothiadiazol-5-yl)-4-(3,5-dimethoxy-4-propoxybenzyl)-5-hydroxy-5-(4-methoxyphenyl)-5H-furan-2-one;
   3-(2,1,3-benzothiadiazol-5-yl)-4-(3,4-dimethoxy-5-isopropoxybenzyl)-5-hydroxy-5-(1,4-benzodioxan-6-yl)-5H-furan-2-one;
   3-(2,1,3-benzothiadiazol-6-methyl-5-yl)-4-(3,4,5-trimethoxybenzyl)-5-hydroxy-5-(4-methoxyphenyl)-5H-furan-2-one;
   3-(2,1,3-benzothiadiazol-5-yl)-4-(4-isopropoxybenzyl)-5-hydroxy-5-(4-methoxyphenyl)-5H-furan-2-one;
   3-(2,1,3-benzothiadiazol-5-yl)-4-(4-hexyloxybenzyl)-5-hydroxy-5-(4-methoxyphenyl)-5H-furan-2-one;
   3-(2,1,3-benzothiadiazol-5-yl)-4-(3,5-dimethoxy-4-isopropoxybenzyl)-5-hydroxy-5-(1,4-benzodioxan-6-yl )-5H-furan-2-one;
   3-(2,1,3-benzothiadiazol-5-yl)-4-(3-methoxy-5-butoxybenzyl)-5-hydroxy-5-(4-methoxyphenyl)-5H-furan-2-one;
   3-(2,1,3-benzothiadiazol-5-yl)-4-(3,4-diisopropoxybenzyl)-5hydroxy-5-(4-rhethoxyphenyl)-5H-furan-2-one;
   3-(2,1,3-benzothiadiazol-5-yl )-4-(3,4,5-trimethoxybenzyl)-5hydroxy-5-(2-fluoro-4-methoxyphenyl)-5H-furan-2-one;
   3-(2,1,3-benzothiadiazol-5-yl)-4-(3,4-dimethoxy-5-isopropoxybenzyl)-5-hydroxy-5-(3-fluoro-4-methoxyphenyl)-5H-furan-2-one;
   3-(2,1,3-benzothiadiazol-5-yl)-4-(3,4-dimethoxy-5-benzyloxybenzyl)-5-hydroxy-5-(4-methoxyphenyl)-5H-furan-2-one;
   3-(2,1,3-benzothiadiazol-5-yl)-4-(3,4,5-trimethoxybenzyl)-5-hydroxy-5-(4-fluoro-2-methoxyphenyl )-5H-furna-2-one;
   3-(2,1,3-benzothiadiazol-5-yl)-4-(3,5-dimethoxy-5-ethoxybenzyl)-5-hydroxy-5-(4-methoxyphenyl)-5H-furan-2-one;
   3-(2,1,3-benzothiadiazol-5-yl)-4-(4-methoxycarbonylbenzyl)-5-hydroxy-5-(4-methoxyphenyl)-5H-furan-2-one;
   3-(2,1,3-benzothiadiazol-5-yl)-4-(3,4-diisopropoxybenzyl)-5-hydroxy-5-(3-fluoro4-methoxyphenyl)-5H-furan-2-one;
   3-(2,1,3-benzothiadiazol-5-yl)-4-(3,4,5-trimethoxybenzyl)-5-hydroxy-5-(4-benzyloxyphenyl)-5H-furan-2-one;
   3-(2,1,3-benzothiadiazol-4-methyl-5-yl)-4-(3,4,5-trimethoxybenzyl)-5-hydroxy-5-(4-methoxyphenyl)-5H-furan-2-one;
   3-(2,1,3-benzothiadiazol-5-yl)-4-(3,5-dimethoxy-4-isobutoxybenzyl)-5-hydroxy-5-(4-methoxyphenyl)-5H-furan-2-one;
   4-(2,1,3-benzothiadiazol-5-yl)-3-(7-methoxy-1,3-benzodioxol-5-yl)-5-hydroxy-5-(4-methoxyphenyl)-5H-furan-2-one;
and the open-chain tautomers;
xvi) the compounds described in WO 9842709
   a) 3-(2,1,3-benzothiadiazol-5-ylmethyl)-1-(4-methoxyphenyl)-8-methyl-3,8-dihydro-3,8-diazacyclopenta[a]indene-2-carboxylic acid;
   b) 3-(2-methoxybenzyl)-1-(4-methoxyphenyl)-8-methyl-3,8-dihydro-3,8-diazacyclopenta[a]indene-2-carboxylic acid;
   c) 3-(2,5-dimethoxybenzyl)-1-(4-methoxyphenyl)-8-methyl-3,8-dihydro-3,8-diazacyclopenta[a]indene-2-carboxylic acid;
   d) 3-(1,3-benzodioxol-5-ylmethyl)-1-(4-methoxyphenyl)-8-methyl-3,8-dihydro-3,8-diazacyclopenta[a]indene-2-carboxylic acid;
   e) 3-(2,1,3-benzothiadiazol-5-ylmethyl)-1-(4-methoxyphenyl)-8-oxa-3-azacyclopenta[a]indene-2-carboxylic acid;
   f) 3-(2,1,3,-benzothiadiazol-5-ylmethyl)-1-(4-methoxyphenyl)-8-thia-3-azacyclopenta[a]indene-2-carboxylic acid;
   g) 3-(2,1,3-benzothiadiazol-5-ylmethyl)-1-(3-carboxymethoxy-4-methoxyphenyl)-8-methyl-3,8-dihydro-3,8-diazacyclopenta[a]-indene-2-carboxylic acid;
   h) 3-(2,1,3-benzothiadiazol-5-ylmethyl)-1-(3-carboxymethoxy-4-methoxyphenyl)-8-oxa-3-azacyclopenta[a]indene-2-carboxylic acid;
   i) 3-(2,1,3-benzothiadiazol-5-ylmethyl)-1-(3-carboxymethoxy4-methoxyphenyl)-8-thia-3-azacyclopenta[a]indene-2-carboxylic acid;
xvii) the compounds described in WO 9905132
   a) 2-(2,1,3-benzothiadiazol-5-yl)-3-(4-cyclopentyloxy-3,5-dimethoxybenzyl)-4-(4-methoxyphenyl)-4-oxo-2-butenoic acid;
   b) 2-(2,1,3-benzothiadiazol-5-yl)-3-(4-cyclopentyloxy-3,5-dimethoxybenzyl)-4-(3-fluoro-4-methoxyphenyl)-4-oxo-2-butenoic acid;
   c) 3-(2,1,3-benzothiadiazol-5-yl)-4-(4-cyclopentyloxy-3,5-dimethoxybenzyl)-5-hydroxy-5-(4-methoxyphenyl)-5H-furan-2-one;
   d) 3-(2,1,3-benzothiadiazol-5-yl)-4-(4-cyclopentyloxy-3,5-dimethoxybenzyl)-5-hydroxy-5-(3-fluoro-4-methoxyphenyl)-5H-furan-2-one;
   e) 3-(2,1,3-benzothiadiazol-5-yl)-4-(3-cyclopentyloxy-4,5-dimethoxybenzyl)-5-hydroxy-5-(3-fluoro-4-methoxyphenyl)-5H-furan-2-one;
   f) 3-(7-methyl-2,1,3-benzothiadiazol-5-yl)-4-(4-cyclopentyloxy-3,5-dimethoxybenzyl)-5-hydroxy-5-(3-fluoro-4-methoxyphenyl)-5H-furan-2-one;
and physiologically acceptable salts and/or solvates thereof for the preparation of a medicament for inhibiting the growth of neoplastic cells.

The invention relates, in particular, to the use of endothelin receptor antagonists selected from the group consisting of
a) 5-dimethylamino-N-(2,1,3-benzothiadiazol-5-yl)naphthalenesulfon-amide;
b) 2-(2,1,3-benzothiadiazol-5-yl)-3-(3-fluoro-4-methoxybenzoyl)-4-(3,4,5-trimethoxyphenyl)but-2-enoic acid;
and physiologically acceptable salts and/or solvates thereof for the preparation of a medicament for inhibiting the growth of neoplastic cells.

For inhibiting the growth of neoplastic cells and for the treatment of tumour diseases, particular preference is given to the use of endothelin receptor antagonists which have high affinity to the ET_{A} receptor.

The invention furthermore relates to the use of the compounds of the formula I and the preferred compounds described above and physiologically acceptable salts and/or solvates thereof for the preparation of a medicament for the treatment and/or prophylaxis of cancer diseases.

The invention furthermore relates to the use of the said compounds, where the cancer diseases are selected from the group consisting of prostate cancer, ovarian carcinoma, intestinal cancer, cervical carcinoma, melanoma and pancreatic cancer.

The invention furthermore relates to the use of the compounds of the formula I and the preferred compounds described above and-physiologically acceptable salts and/or solvates thereof for the preparation of a medicament for the treatment of neoplastic damage.

The invention furthermore relates to the use of the compounds of the formula I and the preferred compounds described above and physiologically acceptable salts and/or solvates thereof for the preparation of a medicament for the treatment of precancerogenic damage.

The term “precancerogenic damage” is taken to mean, for example, benign tumours in the intestine which can result in intestinal cancer.

The term “precancerogenic damage” is taken to mean, in particular, the lesions mentioned in U.S. Pat. No. 5,948,911 at column 4, lines 49-60.

Irregularities in apoptosis (cell death) play a role in the formation of pre-cancerogenic damage.

It is also known that the regulation of apoptosis plays an important role in diseases connected with abnormal cell growth, such as, for example, benign prostate hyperplasia, neurodegenerative diseases, such as, for example, Parkinson's, autoimmune diseases, including multiple sclerosis, and rheumatoid arthritis, or infection diseases, such as AIDS.

The compounds of the formula I modulate apoptosis and are used in the treatment or prophylaxis of cancer diseases.

The invention thus relates to the use of the compounds of the formula I described and the preferred compounds described above and physiologically acceptable salts and/or solvates thereof for the preparation of a medicament for regulating apoptosis in human cells.

The invention furthermore relates to the use of the compounds of the formula I and the preferred compounds described above and/or physiologically acceptable salts thereof for the preparation of pharmaceutical preparations, in particular by non-chemical methods. They can be converted into a suitable dosage form here together with at least one solid, liquid and/or semi-liquid excipient or adjuvant and optionally in combination with one or more further active ingredients.

These preparations can be used as medicaments in human or veterinary medicine. Suitable excipients are organic or inorganic substances which are suitable for enteral (for example oral), parenteral or topical administration and do not react with the novel compounds, for example water, vegetable oils, benzyl alcohols, alkylene glycols, polyethylene glycols, glycerol triacetate, gelatine, carbohydrates, such as lactose or starch, magnesium stearate, talc or Vaseline. Suitable for oral administration are, in particular, tablets, pills, coated tablets, capsules, powders, granules, syrups, juices or drops, suitable for rectal administration are suppositories, suitable for parenteral administration are solutions, preferably oil-based or aqueous solutions, furthermore suspensions, emulsions or implants, and suitable for topical application are ointments, creams or powders. The novel compounds may also be lyophilised and the resultant lyophilisates used, for example, for the preparation of injection preparations. The preparations indicated may be sterilised and/or comprise adjuvants, such as lubricants, preservatives, stabilisers and/or wetting agents, emulsifiers, salts for modifying the osmotic pressure, buffer substances, colorants and flavours and/or a plurality of further active ingredients, for example one or more vitamins. They can furthermore be administered as nasal sprays.

The substances are in general preferably administered here in doses of between about 1 and 500 mg, in particular between 5 and 100 mg per dosage unit. The daily dose is preferably between about 0.02 and 10 mg/kg of body weight. However, the specific dose for each patient depends on a wide variety of factors, for example on the efficacy of the specific compound employed, on the age, body weight, general state of health, sex, on the diet, on the time and method of administration, on the excretion rate, medicament combination and severity of the particular disease to which the therapy applies. Oral administration is preferred.

1. Use of endothelin receptor antagonists selected from the group consisting of
a) the compounds of the formula I described in EP 0733626 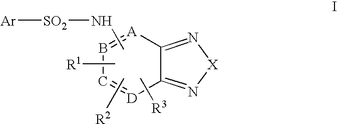
in which
-A=B—C=D- is a —CH═CH—CH═CH— group in which 1 or 2 CH has (have) been replaced by N,
Ar is Ph or naphthyl, each of which is unsubstituted or monosubstituted, disubstituted or trisubstituted by H, Hal, A, alkenyl having up to 6 carbon atoms, Ph, OPh, NO₂, NR⁴R⁵, NHCOR⁴, CF₃, OCF₃, CN, OR⁴, COOR⁴, (CH₂)ₙCOOR⁴, (CH₂)ₙNR⁴R⁵, —N═C═O or NHCONR⁴R⁵,
R¹,R²
and R³ are each, independently of one another, absent, H, Hal, A, CF₃, NO₂, NR⁴R⁵, CN, COOR⁴ or NHCOR⁴,
R⁴ and R⁵ are each, independently of one another, H or A, or together are alternatively —CH₂—(CH₂)ₙ—CH₂—,
A is alkyl having from 1 to 6 carbon atoms,
Ph is phenyl,
X is O or S,
Hal is F, Cl, Br or I,
n is 1, 2 or 3,
and the salts thereof;
b) the compounds of the formula I described in EP 0758650 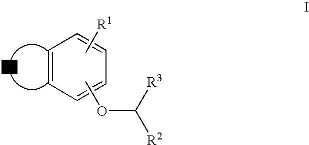
in which
X is a saturated, fully unsaturated or partially unsaturated 3-to 4-membered alkylene chain, in which from 1 to 3 carbon atoms may be replaced by N and/or 1 or 2 carbon atoms may be replaced by 1-2 O atoms and/or 1-2 S atoms, but where at most up to 3 carbon atoms are replaced and where, in addition, a monosubstitution, disubstitution or trisubstitution of the alkylene chain and/or of a nitrogen located therein by A, R⁸ and/or NR⁴R⁴ may occur, and where, furthermore, one CH₂ group in the alkylene chain may also be replaced by a C═O group,
A is alkyl having 1-6 carbon atoms, in which one or two CH₂ groups may be replaced by O or S atoms or by —CR⁴═CR^{4′}— groups and in addition 1-7 H atoms may be replaced by F,
R¹ is H or A,
R² is COOR⁴, CN, 1H-tetrazol-5-yl or CONHSO₂R⁸,
R³ is Ar,
R⁴ and R^{4′} are each, independently of one another, H, alkyl having from 1 to 6 carbon atoms or benzyl,
Ar is phenyl or naphthyl, each of which is unsubstituted or monosubstituted, disubstituted or trisubstituted by R⁵, R⁶ or R⁷, or a 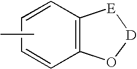
group which is unsubstituted or monosubstituted or disubstituted by in the phenyl moiety R⁵ or R⁶,
R⁵, R⁶
and R⁷ are each, independently of one another, R⁴, OR⁴, Hal, CF₃, OCF₃, OCHF₂, OCH₂F, NO₂, NR⁴R^{4′}, NHCOR⁴, CN, NHSO₂R⁴, COOR⁴, COR⁴, CONHSO₂R⁸, O(CH₂)ₙR², OPh, O(CH₂)ₙOR⁴ or S(O)ₘR⁴,
R⁸ is phenyl or naphthyl, each of which is unsubstituted or monosubstituted, disubstituted or trisubstituted by A, OR¹, NR⁴R^{4′} or Hal,
E is CH₂ or O,
D is carbonyl or [C(R⁴R^{4′})]ₙ,
Hal is F, Cl, Br or I,
m is 0, 1 or 2,
n is 1 or 2,
and salts thereof;
c) the compounds of the formula I described in EP 0755934 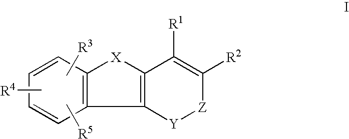
in which
—Y-Z- is —NR⁷—CO—, —N═C(OR⁷)— or —N═CR⁸—,
R¹ is Ar,
R² is COOR⁶, CN, 1H-tetrazol-5-yl or CONHSO₂Ar,
R³, R⁴
and R⁵ are each, independently of one another, R⁶, OR⁶, S(O)ₘR⁶, Hal, NO₂, NR⁶R^{6′}, NHCOR⁶, NHSO₂R⁶, OCOR⁶, COOR⁶ or CN,
R⁶ and R^{6′} are each, independently of one another, H, alkyl having from 1 to 6 carbon atoms, benzyl or phenyl,
R⁷ is (CH₂)ₙAr,
R⁸ is Ar or OAr,
Ar is phenyl which is unsubstituted or monosubstituted, disubstituted or trisubstituted by R⁹, R¹⁰ or R¹¹, or
unsubstituted naphthyl, or a 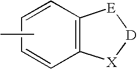
group which is unsubstituted or monosubstituted or disubstituted in the phenyl moiety by R⁹ or R¹⁰, or a 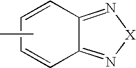
group which is unsubstituted or monosubstituted or disubstituted in the cyclohexadienyl moiety by R⁹ or R¹⁰,
R⁹ R¹⁰
and R¹¹ are each, independently of one another, R⁶, OR⁶, Hal, CF₃, OCF₃, OCHF₂, OCH₂F, NO₂, NR⁶R^{6′}, NHCOR⁶, CN, NHSO₂R⁶, COOR⁶, COR⁶, CONHSO₂Ar, O(CH₂)ₙR², O(CH₂)ₙOR⁶ or S(O)ₘR⁶,
E is CH₂,S or O,
D is carbonyl or [C(R R^{6′})]ₙ,
Hal is F, Cl, Br or I,
X is O or S,
m is 0, 1 or 2,
n is 1 or 2,
and salts thereof;
d) the compounds of the formula I described in EP 0757039 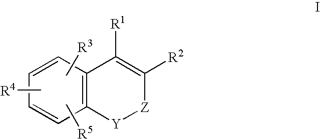
in which
—Y-Z- is —NR⁷—CO—, —N═C(OR⁷)— or —N═CR⁸—,
R¹ is Ar,
R² is COOR⁶, (CH₂)ₙCOOR⁶, CN, 1H-tetrazol-5-yl or CONHSO₂Ar,
R³, R⁴
and R⁵ are each, independently of one another, R⁶, OR⁶, S(O)ₘR⁶, Hal, NO₂, NR⁶R^{6′}, NHCOR⁶, NHSO₂R⁶, OCOR⁶, COR⁶, COOR6 or CN, where R³ and R⁴ together can alternatively be an O(CH₂)ₙ) group,
R⁶ and R^{6′} are each, independently of one another, H, alkyl having from 1 to 6 carbon atoms, benzyl or phenyl,
R⁷ is (CH₂)ₙAr,
R⁸ is Ar or OAr,
Ar is phenyl which is unsubstituted or monosubstituted, disubstituted or trisubstituted by R⁹, R¹⁰ or R¹¹, or
unsubstituted naphthyl, or a 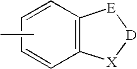
group which is unsubstituted or monosubstituted or disubstituted in the phenyl moiety by R⁹ or R¹⁰, or a 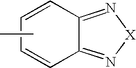
group which is unsubstituted or monosubstituted or disubstituted in the cyclohexadienyl moiety by R⁹ or R¹⁰,
R⁹, R¹⁰
and R¹¹ are each, independently of one another, R⁶, OR⁶ Hal, CF₃, OCF₃, OCHF₂, OCH₂F, NO₂, NR⁶R^{6′}, NHCOR⁶, CN, NHSO₂R⁶, COOR⁶, COR⁶, CONHSO₂Ar, O(CH₂)ₙR², O(CH₂)ₙOR⁶ or S(O)ₘR^{l ,}
E is CH₂, S or O,
D is carbonyl or [C(R⁶R^{6′})]ₙ,
X is O or S,
Hal is F, Cl, Br or I,
m is 0, 1 or 2,
n is 1, 2 or 3,
and salts thereof;
e) the compounds of the formula I described in EP 0796250 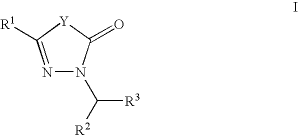
in which
Y is —C(R⁴R^{4′})—C(R⁴R^{4′})—, —CR⁴═CR^{4′}— or —C(R⁴R^{4′})—S—,
R¹ is Het, Ar, R³ or R⁴,
R² is Ar or a 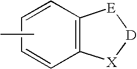
group which is unsubstituted or monosubstituted or disubstituted in the phenyl moiety by A, R³, OR⁴, NH₂, NHA, NA₂, NO₂, CN, Hal, NHCOR⁴, NHSO₂R⁴, COOR⁴, COR⁴, CONHSO₂R⁶, O(CH₂)ₙR³, OPh, O(CH₂)ₙOR⁴ or S(O)ₘR⁴, or a 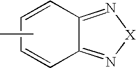
group which is unsubstituted or monosubstituted or disubstituted in the cyclohexadienyl moiety by A, R³, OR⁴, NH₂, NHA, NA₂, NO₂, CN, Hal, NHCOR⁴, NHSO₂R⁴, COOR⁴, COR⁴, CONHSO₂R⁶, O(CH₂)ₙR³, OPh, O(CH₂)ₙOR⁴ or S(O)ₘR⁴,
R³ is CN, COOH, COOA, CONHSO₂R⁵or 1H-tetrazol-5-yl,
R⁴ and R^{4′} are each, independently of one another, H, A, or phenyl or benzyl, each of which is unsubstituted or monosubstituted by alkoxy,
R⁵ is A or Ar,
R⁶ is phenyl or naphthyl, each of which is unsubstituted or monosubstituted, disubstituted or trisubstituted by A, OR⁵, NH₂, NHA, NA₂, NO₂, CN or Hal,
A is alkyl having 1-6 carbon atoms, in which one or two CH₂ groups may be replaced by O or S atoms or by —CR⁴═CR⁴— groups and in addition 1-7 H atoms may be replaced by F,
or benzyl,
Ar is phenyl or naphthyl, each of which is unsubstituted or monosubstituted, disubstituted or trisubstituted by A, OR⁴, NH₂, NHA, NA₂, NO₂, CN, Hal, NHCOR⁴, NHSO₂R⁴, COOR⁴, COR⁴, CONHSO₂R⁶, O(CH₂)ₙR³, OPh, O(CH₂)ₙOR⁴ or S(O)ₘR⁴,
Het is a monocyclic or bicyclic saturated, unsaturated or aromatic heterocyclic radical having from 1 to 4 N, O and/or S atoms, bonded via N or C, which may be unsubstituted or monosubstituted, disubstituted or trisubstituted by Hal, A, R³, NH₂, NHA, NA₂, CN, NO₂ and/or carbonyl oxygen,
D is carbonyl or [C(R⁴R^{4′})]ₙ,
E is CH₂, S or O,
Hal is F, Cl, Br or I,
X is O or S,
m is 0, 1 or 2,
n is 1 or 2,
and salts thereof;
f) the compounds of the formula I described in WO 9719077 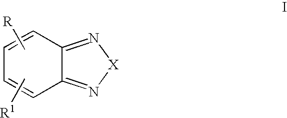
in which
R is 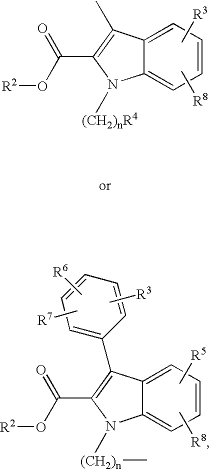
X is O or S,
R is H, Hal, OH, OA, A, alkylene-O-A, NO₂, NH₂, NH-acyl, SO₂NH₂, SO₃-A, SO₂NHA, CN or formyl,
R² is H or A,
R³, R⁵, R⁶,
R⁷ and R⁸ are each, independently of one another, H, Hal, OH, OA, O-alkylene-R⁴, A, S-A, NO₂, NH₂, NHA, NA₂, NH-acyl, NHSO₂A, NHSO₂R⁴, NASO₂A, NASO₂—R⁴, NH(CO)NH₂, NH(CO)NHA, formyl, NH(CO)NH-phenyl, NHCOOA, NA-acyl, NHR⁴, NHCOOR⁴, NHCOO-benzyl, NHSO₂-benzyl, NHCOO-alkylene-OA, NH(CO)NA₂, N-piperidinyl-CO—NH, N-pyrrolidinyl-CONH, O(CH₂)ₙCOOR², O(CH₂)ₙOR , CH₂OH or CH₂OA,
R³ and R⁶ together are alternatively —O—CH₂—O—, —O—CH₂—CH₂—O—, —O—CH₂—CH₂—, O—CF₂—O— or —O—CF₂—CF₂—O—,
R⁴ is phenyl which is unsubstituted or monosubstituted or polysubstituted by R³ and/or R⁶,
A is alkyl having 1-6 carbon atoms,
Hal is fluorine, chlorine, bromine or iodine,
n is 1 or 2,
and salts thereof;
g) the compounds of the formula I described in WO 9730982 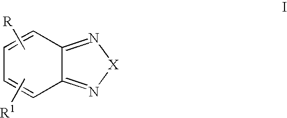
in which
R is 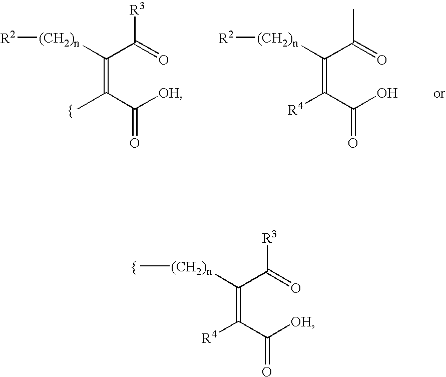
X is O or S,
R¹ is H, Hal, OH, OA, A, alkylene-O-A, NO₂, NH₂, NH-acyl, SO₂NH₂, SO₃-A, SO₂NHA, CN or formyl,
R², R³
and R⁴ are each, independently of one another, a phenyl group which is unsubstituted or monosubstituted or polysubstituted by Hal, OH, OA, O-alkylene-R⁵, A, S-A, SOA, SO₂A, SOR⁵, SO₂R⁵, NO₂, NH₂, NHA, NA₂, NH-acyl, NHSO₂A, NHSO₂R⁵, NASO₂A, NASO₂—R⁵, NH(CO)NH₂, NH(CO)NHA, formyl, NH(CO)NHR⁵, NHCOOA, NA-acyl, NHCOOCH₂R⁵, NHSO₂CH₂R⁵, NHCOO-alkylene-OA, NH(CO)NA₂, 1-piperidinyl-CO-NH, 1-pyrrolidinyl-CONH, O(CH₂)ₙCOOA, O(CH₂)ₙCOOH, O(CH₂)ₙOH, O(CH₂)ₙOA, CH₂OH, CH₂OA, COOH, COOA, CH₂COOH or CH₂COOA,
a 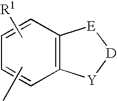
group or a 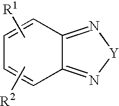
group, where is additionally A or cycloalkyl,
R⁵ is a phenyl group which is unsubstituted or monosubstituted or polysubstituted by Hal, OH, OA, A, S-A, NO₂, NH₂, NHA, NA₂, NH-acyl, NHSO₂A, NASO₂A, NH(CO)NH₂, NH(CO)NHA, formyl, NHCOOA, NA-acyl, NHCOO-alkylene-OA, NH(CO)NA₂, N-piperidinyl-CO—NH, N-pyrrolidinyl-CON H, O(CH₂)ₙCOOA, O(CH₂)ₙCOOH, O(CH₂)ₙOH, O(CH₂)ₙOA, CH₂OH, CH₂OA, COOH, COOA, CH₂COOH or CH₂COOA,
A is alkyl having 1-6 carbon atoms, in which one or two CH₂ groups may be replaced by O or S atoms or by —CR⁶═CR⁶— groups and/or 1-7 H atoms may be replaced by F,
D is carbonyl or [C(R⁶R^{6′})]ₘ,
E is CH₂, S or O,
Y is O or S,
R⁶ and R^{6′} are each, independently of one another, H, F or A,
Hal is fluorine, chlorine, bromine or iodine,
n is 1 or 2, and
m is 1 or 2,
or a tautomeric cyclised form, and the (E)-isomers and the salts of all isomers;
h) the compounds of the formula I described in WO 9730996 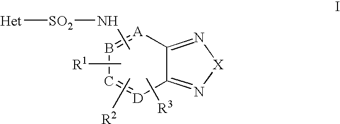
in which
-A=B—C=D- is a —CH═CH—CH═CH— group, in which, in addition, 1 or 2 CH may be replaced by N,
Het is a monocyclic or bicyclic saturated, unsaturated or aromatic heterocyclic radical having from 1 to 4 N, O and/or S atoms which is unsubstituted or substituted by -Z-R⁶,
R¹, R²
and R³ are each, independently of one another, absent, H, Hal, A, CF₃, NO₂, NR⁴R⁵, CN, COOR⁴ or NHCOR⁴,
R⁴ and R⁵ are each, independently of one another, H or A, or together are alternatively —CH₂—(CH₂)ₙ—CH₂—,
R⁶ is a phenyl radical, a benzothiadiazol-5-yl radical or a benzoxadiazol-5-yl radical, each of which is unsubstituted or monosubstituted, disubstituted or trisubstituted by R⁷, R⁸ and/or R⁹,
R⁷, R⁸
and R⁹ are each, independently of one another, A, O-A, CN, COOH, COOA, Hal, formyl or —CO-A, and R⁷ and R⁸ together are alternatively —O—(CH₂)ₘ—O—,
A is alkyl having from 1 to 6 carbon atoms,
X is O or S,
Z is —CO—, —CONH—, —CO—(CH₂)ₙ—, —CH═CH—, —(CH₂)ₙ—, —CONHCO—,—NHCONH—, —NHCOO —, —O—COHN—, —CO—O— or —O—CO—,
Hal is F, Cl, Br or I,
m is 1 or 2, and
n is 1, 2 or 3,
and salts thereof;
i) the compounds of the formula I described in DE 19609597 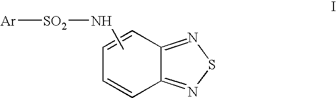
in which
Ar is naphthyl which is monosubstituted by NH₂, NHA or NA₂, and
A is alkyl having from 1 to 6 carbon atoms,
and physiologically acceptable salts thereof;
j) the compounds of the formula I described in DE 19612101 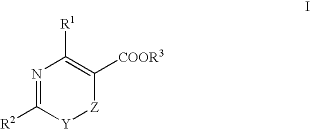
in which
—Y-Z- is —NR⁴—Co or —N═CR⁵—,
R¹ is Ar,
R² is H, alkyl having 1-6 carbon atoms which is unsubstituted or monosubstituted, disubstituted or trisubstituted by OR³ or Hal, or (CH₂)ₘPh or (CH₂)ₘ-cycloalkyl, each of which is unsubstituted or monosubstituted, disubstituted or trisubstituted by R³, OR³ or Hal,
R³ and R^{3′} are each, independently of one another, H, alkyl having 1-6 carbon atoms or benzyl,
R⁴ is CH₂Ar,
^{R5} is OCH₂Ar,
Ar is phenyl which is unsubstituted or monosubstituted, disubstituted or trisubstituted by R⁶, R⁷ or R⁸, or a 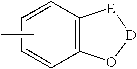
group which is unsubstituted or monosubstituted in the phenyl moiety by R⁶, or a 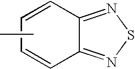
group which is unsubstituted or monosubstituted in the cyclohexadienyl moiety by R⁶,
E is CH₂ or O,
D is carbonyl or (CH₂)ₙ,
E and D together are alternatively CH═CR⁹,
R⁶ and R^{6′} are each, independently of one another, R³, OR³or Hal,
R⁷ is R³, OR³, Hal, NO₂, NH₂, NHR³, NR³R^{3′}, NHCOR³, COOR³, O(CH₂)ₙR³ or O(CH₂)ₙOR³,
R⁸ is Ph which is unsubstituted or monosubstituted, disubstituted or trisubstituted by R³, OR³, Hal, NO₂, NH₂, NHR⁶, NR⁶R^{6′}, NHCOR³ or COOR³,
R⁹ is H, OH, CH₂OH or COOR³,
Hal is F, Cl, Br or I,
Ph is phenyl,
m is 0 or 1,
n is 1 or 2,
and salts thereof;
k) the compounds of the formula I described in WO 9827091 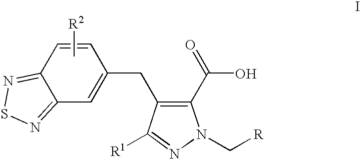
in which
R is phenyl which is unsubstituted or monosubstituted, disubstituted or trisubstituted by R³, R⁴ or R⁵ or is 2,1,3-benzothiadiazolyl which is unsubstituted or monosubstituted by R²,
R¹ is A, in which 1-7 H atoms may be replaced by F, or is —S-A or —O-A, or phenyl or -alkylene-phenyl, each of which is unsubstituted or monosubstituted by R³, or thienyl which is unsubstituted or monosubstituted by R³,
R² is A, F, Cl, Br or —O-A,
R³, R⁴
and R⁵ are each, independently of one another, A, —O-A, —S-A, —O-alkylene-COOH, -alkylene-COOH or COOH,
R³ and R⁴ together are alternatively —O—CH₂—O—, and
A is alkyl having 1-7 carbon atoms,
and salts thereof;
I) the compounds of the formula I described in WO 9827077 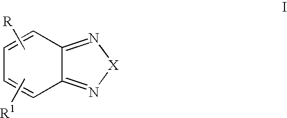
in which
R is 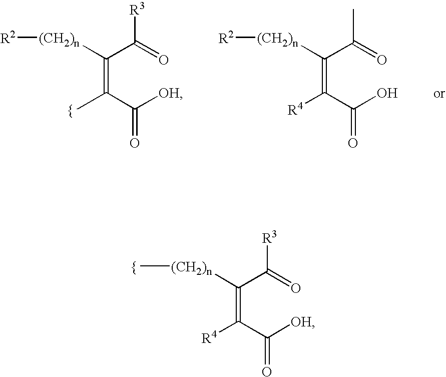
X is O or S,
R¹ is H, Hal, OH, OA, A, alkylene-O-A, NO₂, NH₂, NH-acyl, SO₂NH₂, SO₃-A, SO₂NHA, CN or formyl,
R² R³
and R⁴ are each, independently of one another, a phenyl group which is unsubstituted or monosubstituted or polysubstituted by R⁷, where R is additionally A or cycloalkyl,
a 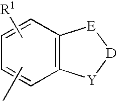
group or a 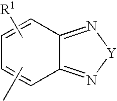
group, with the proviso that at least one of the radicals R², R³ or R⁴ is an R⁸ radical which is unsubstitdted or monosubstituted or polysubstituted by R⁷,
R⁵ is a phenyl group which is unsubstituted or monosubstituted or polysubstituted by Hal, OH, OA, A, S-A, NO₂, NH₂, NHA, NA₂, NH-acyl, NHSO₂A, NASO₂A, NH(CO)NH₂, NH(CO)NHA, formyl, NHCOOA, NA-acyl, NHCOO-alkylene-OA, NH(CO)NA₂, N-piperidinyl-CO—NH, N-pyrrolidinyl-CONH, O(CH₂)ₙCOOA, O(CH₂)ₙCOOH, O(CH₂)ₙOH, O(CH₂)ₙOA, CH₂OH, CH₂OA, COOH, COOA, CH₂COOH or CH₂COOA,
A is alkyl having 1-6 carbon atoms, in which one or two CH₂ groups may be replaced by O or S atoms or by —CR⁶═CR⁶— groups and/or 1-7 H atoms may be replaced by F,
D is carbonyl or [C(R⁶R^{6′})]ₘ,
E is CH₂, S or O,
Y is O or S,
R⁶ and R^{6′} are each, independently of one another, H, F or A,
R⁷ is Hal, OH, OA, O-alkylene-R⁵, A, S-A, S—OA, SO₂A, S—OR⁵, SO₂R⁵, NO₂, NH₂, NHA, NA₂, NH-acyl, NHSO₂A, NHSO₂R⁵, NASO₂A, NASO₂—R⁵, NH(CO)NH₂, NH(CO)NHA, formyl, NH(CO)NHR⁵, NHCOOA, NA-acyl, NHCOOCH₂R⁵, NHSO₂CH₂R⁵, NHCOO-alkylene-OA, NH(CO)NA₂, 1-piperidinyl-CO—NH, 1-pyrrolidinyl-CONH, O(CH₂)ₙCOOA, O(CH₂)ₙCOOH, O(CH₂)ₙOH, O(CH₂)ₙOA, CH₂OH, CH₂OA, COOH, COOA, CH₂COOH or CH₂COOA,
R⁸ is a 5-7 membered heterocyclic radical having 1-4 N, O and/or S atoms or
a 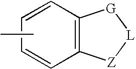
group,
G and Z are each, independently of one another, —CH═, N, O or S,
L is —CH═, —CH═CH— or —CH₂—CH₂—CH₂—,
Hal is fluorine, chlorine, bromine or iodine,
n is 0, 1 or 2, and
m is 1 or 2,
or a tautomeric cyclised form, and the (E)-isomers and salts of all isomers;
m) the compounds of the formula I described in WO 9841515 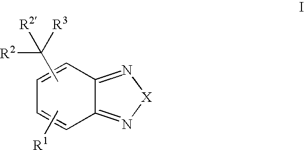
in which
X is O or S,
R¹ is H, Hal, OH, OA, A, NO₂, NH₂, NHA, NAA′, NHCOR⁴, NHCOR⁶, NHSO₂R⁴, NHSO₂R⁶, S(O)ₘR⁶, SO₃H, SO₂NR⁴R^{4′} or formyl,
R² and R^{2′} are each, independently of one another, A, (CH₂)ₙAr, (CH₂)ₙHet, CH₂COAr, CH₂COHet or OAr,
R^{2′}0 is additionally also H,
R³ is COOR⁴, CN, 1H-tetrazol-5-yl or CONHSO₂R⁵,
R⁴ and R^{4′} are each, independently of one another, H or A,
R⁵ is A or Ar,
R⁶ is phenyl or naphthyl, each of which is unsubstituted or monosubstituted, disubstituted or trisubstituted by A, NH₂, NHA, NAA′, NO₂, CN or Hal,
R⁷ and R^{7′} are each, independently of one another, H or alkyl having 1-6 carbon atoms,
A and A′ are each, independently of one another, alkyl having 1-6 carbon atoms, in which one or two CH₂ groups may be replaced by O or S atoms or by —CR⁷═CR^{7′} groups and/or 1-7 H atoms may be replaced by F,
or benzyl,
Ar is phenyl or naphthyl, each of which is unsubstituted or monosubstituted, disubstituted or trisubstituted by A, OR⁴, NH₂, NHA, NAA′, NO₂, CN, Hal, NHCOR⁴, NHCOR⁶, NHSO₂R⁴, NHSO₂R⁶, COOR⁴, OPh, CONH₂, CONHA, CONAA′, COR⁴, CONHSO₂R⁴, CONHSO₂R⁶, O(CH₂)ₙCOOR⁴, O(CH₂)ₙOR⁴, SO₃H, SO₂NR⁴R^{4′}, S(O)ₘR⁶ or S(O)ₘR⁴,
Het is a monocyclic or bicyclic saturated, unsaturated or aromatic heterocyclic radical having 1-4 N, O and/or S atoms, bonded via N or C, which may be unsubstituted or monosubstituted, disubstituted or trisubstituted by Hal, A, R³, NH₂, NHA, NAA′, NO₂ and/or ═O,
Hal is fluorine, chlorine, bromine or iodine,
m is 0, 1 or 2, and
n is 1 or 2,
where, if R² is CH₂COAr and R^{2′} is H, R³ is not COOA,
and salts thereof;
n) the compounds of the formula I described in WO 9841521 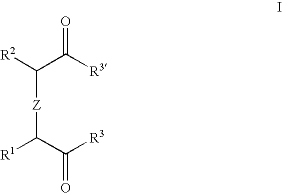
in which
Z is a single or double bond,
R¹ is a 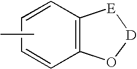
group which is unsubstituted or monosubstituted in the phenyl moiety by R⁷, or a 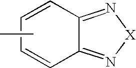
group which is unsubstituted or monosubstituted in the cyclohexadienyl moiety by R⁷,
R² is A, Ar—(CH₂)ₘ, cycloalkyl-(CH₂)ₘ, Het-(CH₂)ₘ or R¹—(CH₂)ₘ,
R³ and R^{3′} are each, independently of one another, OR⁴, NHSO₂R⁵, NH₂, NHA or NAA′,
R³ and R^{3′} together are alternatively —O—, forming a cyclic anhydride,
R⁴ and R^{4′} are each, independently of one another, H or A,
R⁵ is A or Ar,
R⁶ is phenyl or naphthyl, each of which is unsubstituted or monosubstituted, disubstituted or trisubstituted by A, NH₂, NHA, NAA′, NO₂, CN or Hal,
R⁷ is A, COOR⁴, CN, 1H-tetrazol-5-yl, CONHSO₂R⁵, Hal, OR⁴, NO₂, NH₂, NHA, NAA′, NHCOR⁴, NHCOR⁶, NHSO₂R⁴, NHSO₂R⁶, S(O)ₖR⁴, S(O)ₖR⁶, SO₂NR⁴R^{4′} or formyl,
R⁸ and R^{8′} are each, independently of one another, H or alkyl having 1-6 carbon atoms,
E is CH₂or O,
D is carbonyl or (CR⁴R^{4′})ₙ,
E and D together are alternatively CR⁴═R^{4′},
X is S or O,
A and A′ are each, independently of one another, alkyl having 1-6 carbon atoms, in which one or two CH₂ groups may be replaced by O or S atoms or by —CR⁸═CR^{8′}— groups and/or 1-7 H atoms may be replaced by F,
or benzyl,
Ar is phenyl or naphthyl, each of which is unsubstituted or monosubstituted, disubstituted or trisubstituted by A, OR⁴, NH₂, NHA, NAA′, NO₂, CN, Hal, NHCOR⁴, NHCOR⁶, NHSO₂R⁴, NHSO₂R⁶, COOR⁴, OPh, CONH₂, CONHA, CONAA′, COR⁴, CONHSO₂R⁴, CONHSO₂R⁶, O(CH₂)ₙCOOR⁴, O(CH₂)ₙOR⁴, SO₂NR⁴R^{4′}, S(O)ₖR⁶ or S(O)ₖR⁴,
Het is a monocyclic or bicyclic saturated, unsaturated or aromatic heterocyclic radical having 1-4 N, O and/or S atoms, bonded via N or C, which may be unsubstituted or monosubstituted, disubstituted or trisubstituted by Hal, A, COOR⁴, CN, 1H-tetrazol-5-yl, CONHSO₂R⁵, NH₂, NHA, NAA′, NO₂ and/or ═O,
Hal is fluorine, chlorine, bromine or iodine,
k is 0, 1 or 2,
m is 0,1 or 2, and
n is 1 or 2,
and the (Z)- and (E)-isomers and the salts of all isomers;
o) the compounds of the formula I described in WO 9842702 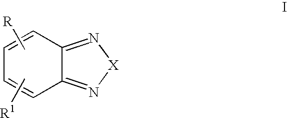
in which
R is 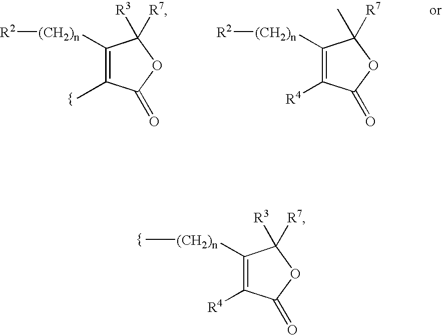
X and Y are each, independently of one another, O or S,
R¹ is H, Hal, OH, OA, A, alkylene-O-A, NO₂, NH₂, NH-acyl, SO₂NH₂, SO₂-A, SO₂N HA, CN or formyl,
R², R³
and R⁴ are each, independently of one another, a phenyl group which is unsubstituted or monosubstituted or polysubstituted by Hal, OH, OA, O-alkylene-R⁵, A, S-A, S—OA, SO₂A, S—OR⁵, SO₂R, NO₂, NH₂, NHA, NA₂, —NH-acyl, NHSO₂A, NHSO₂R⁵, NASO₂A, NASO₂—R⁵, NH(CO)NH₂, NH(CO)NHA, formyl, NH(CO)NHR⁵, NHCOOA, NA-acyl, NHCOOCH₂R⁵, NHSO₂CH₂R⁵, NHCOO-alkylene-OA, NH(CO)NA₂, 1-piperidinyl-CO-NH, 1-pyrrolidinyl-CONH, O(CH₂)ₙCOOA, O(CH₂)ₙCOOH, O(CH₂)ₙOH, O(CH₂)ₙOA, CH₂OH, CH₂OA, COOH, COOA, CH₂COOH or CH₂COOA,
a 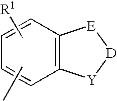
group or a 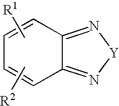
group, where is additionally A or cycloalkyl,
R⁵ is a phenyl group which is unsubstituted or monosubstituted or polysubstituted by Hal, OH, OA, A, S-A, NO₂, NH₂, NHA, NA₂, NH-acyl, NHSO₂A, NASO₂A, NH(CO)NH₂, NH(CO)NHA, formyl, NHCOOA, NA-acyl, NHCOO-alkylene-OA, NH(CO)NA₂, N-piperidinyl-CO—NH, N-pyrrolidinyl-CONH, O(CH₂)ₙCOOA, O(CH₂)ₙCOOH, O(CH₂)ₙOH, O(CH₂)ₙOA, CH₂OH, CH₂OA, COOH, COOA, CH₂COOH or CH₂COOA,
A is alkyl having 1-6 carbon atoms, in which one or two CH₂ groups may be replaced by O or S atoms or by —CR⁶═CR^{6′}— groups and/or 1-7 H atoms may be replaced by F,
D is carbonyl or [C(R⁶R^{6′})]ₘ,
E is CH₂, S or O,
R⁶ and R^{6′} are each, independently of one another, H, F or A,
R⁷ is —O—C(═Y)—NH—R⁸,
R⁸ is alkyl having 1-10 carbon atoms which is unsubstituted or monosubstituted or disubstituted by R⁹ and in which 1-2 carbon atoms may be replaced by O and/or S and/or may be substituted by ═O,
or
cycloalkyl, in which 1-2 carbon atoms may be replaced by N, O and/or S,
R⁹ is phenyl which is unsubstituted or monosubstituted or disubstituted by Hal,
naphthyl, A-O—C(═O)— or Hal,
Hal is fluorine, chlorine, bromine or iodine,
n is 0, 1 or 2, and
m is 1 or 2,
and salts thereof;
p) the compounds of the formula I described in WO 9842709 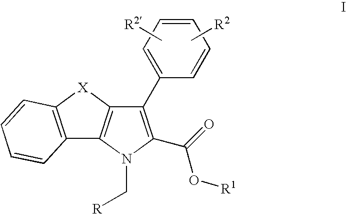
in which
X is N—R³, O or S,
R is 2,1,3-benzothiadiazol-4-or 5-yl or 2,1-benzoisothiazol-5-or 6-yl, each of which is unsubstituted or monosubstituted or disubstituted by R² and/or R^{2′},
or
phenyl which is unsubstituted or monosubstituted, disubstituted or trisubstituted by R² and/or R^{2′},
R¹ is H or A,
R² and R^{2′} are each, independently of one another, H, A, OH, OA, Hal, OCF₃, OCHF₂, —O—CO-A, —O-alkylene-COOR¹, —O-alkylene-CH₂-OR¹,
or
OCH₂-phenyl or —O—CO-phenyl, each of which is unsubstituted or monosubstituted or disubstituted in the phenyl moiety by R⁴ and/or R^{4′},
R² and R^{2′} together are alternatively —OCH₂O—, —OCH₂CH₂O— or —OCH₂CH₂—,
R³ is H, A, alkylene-O-A, —CO—OA, or alkylene-phenyl which is unsubstituted or monosubstituted or disubstituted in the phenyl moiety by R⁴ and/or R^{4′},
R⁴ and R^{4′} are each, independently of one another, H, A, OH, OA, Hal, COOR¹ or CH₂OR¹,
A is alkyl having 1-6 carbon atoms,
Hal is fluorine, chlorine, bromine or iodine,
and salts thereof;
q) the compounds of the formula I described in WO 9905132 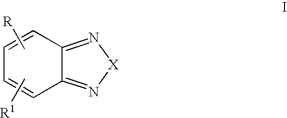
in which
R is 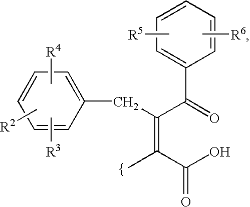
X is O or S,
R¹ is H, Hal, OA or A,
R², R³, R⁵ and R⁶ are each, independently of one another, H, Hal, A, OA or R⁴,
R⁴ is —O—(CH₂)ₙ-Cy,
Cy is cycloalkyl having 3-8 carbon atoms,
A is alkyl having 1-6 carbon atoms, in which one or two CH₂ groups may be replaced by O or S atoms or by —CR⁵═CR^{5′}— groups and/or 1-7 H atoms may be replaced by F,
R⁵ and R^{5′} are each, independently of one another, H, F or A,
Hal is fluorine, chlorine, bromine or iodine,
n is 0, 1 or 2,
or a tautomeric cyclised form, and the (E)-isomers and the salts of all isomers,
for the preparation of a medicament for inhibiting the growth of neoplastic cells.

2. Use of endothelin receptor antagonists selected from the group consisting of
i) the compounds described in EP 0733626
a) 5-bromo-2-ethyl-N-(2,1,3-benzothiadiazol-5-yl)benzenesulfonamide;
b) 2,5-dichloro-N-(2,1,3-benzothiadiazol-5-yl)benzenesulfonamide;
c) 5-bromo-2-propyl-N-(2,1,3-benzothiadiazol-5-yl)benzenesulfonamide;
d) 5-dimethylamino-N-(2,1,3-benzothiadiazol-5-yl)naphthalenesulfonamide;
e) 5-dimethylamino-N-[6-methyl-(2,1,3-benzothiadiazol-5-yl)]-naphthalenesulfonamide;
f) 5-dimethylamino-N-[4-bromo-(2,1,3-benzothiadiazol-5-yl)]-naphthalenesulfonamide;
g) 5-dimethylamino-N-(2,1,3-benzothiadiazol-4-yl)naphthalenesulfonamide;
h) 5-dimethylamino-N-([1,2,5]-oxadiazole-[3,4-b]-pyridin-6-yl)-naphthalenesulfonamide;
i) 5-dimethylamino-N-(1,2,5-benzoxadiazol-5-yl)-1-naphthalenesulfonamide;
j) 5-dimethylamino-N-(6-bromo-7-methyl-1,2,5-benzoxadiazol-5-yl)-1-naphthalenesulfonamide;
k) 2-phenyl-N-(2,1,3-benzothiadiazol-5-yl)benzenesulfonamide;
ii) the compounds described in EP 0758650
a) 2-(1,3-benzodioxol-5-yl)-2-(1,3-dihydro-1,3-dioxoisoindol-5-yloxy)acetic acid;
b) 2-(1,3-benzodioxol-5-yl)-2-(1,3-dihydro-1,3-dioxoisoindol-5-yloxy)-N-(4-tert-butylphenylsulfonyl)acetamide;
c) 2-(1,3-benzodioxol-5-yl)-2-(1,3-dihydro-1,3-dioxoisoindol-5-yloxy)-N-(4-isopropylphenylsulfonyl)acetamide;
d) 2-(1,3-benzodioxol-5-yl)-2-(7-propylquinolin-8-yloxy)acetic acid;
e) 2-(1,3-benzodioxol-5-yl)-2-(7-propylquinolin-8-yloxy)-N-(4-tert-butylphenylsulfonyl)acetamide;
f) 2-(1,3-benzodioxol-5-yl)-2-(6-propylindol-7-yloxy)acetic acid;
g) 2-(1,3-benzodioxol-5-yl)-2-(1-methyl-2-propylbenzim idazol-4-yloxy)acetic acid;
iii) the compounds described in EP 0755934
a) 1,2-dihydro-1-(2-methoxybenzyl)-4-(4-methoxyphenyl)-2-oxobenzofuro[3,2-b]pyridine-3-carboxylic acid;
b) 2-(2-methoxybenzyloxy)-4-(4-methoxyphenyl)benzofuro[3,2-b]-pyridine-3-carboxylic acid;
c) 4-(1,4-benzodioxan-6-yl)-1,2-dihydro-1-(2-methoxybenzyl)-2-oxobenzofuro[3,2-b]pyridine-3-carboxylic acid;
d) 2-(2-methoxyphenoxy)-4-(4-methoxyphenyl)benzofuro[3,2-b]-pyridine-3-carboxylic acid;
e) 4-(1,4-benzodioxan-6-yl)-1,2-dihydro-1-(2-methoxybenzyl)-2-oxo-3-(1H-tetrazol-5-yl)benzofuro[3,2-b]pyridine;
f) 1,2-dihydro-1-(2,3-methylenedioxybenzyl)-4-(4-methoxyphenyl)-2-oxobenzofuro[3,2-b]pyridine-3-carboxylic acid;
g) 1,2-dihydro-1-(2,3-methylenedioxybenzyl)-7-methyl-4-(4-trifluoromethoxyphenyl)-2-oxobenzofuro[3,2-b]pyridine-3-carboxylic acid;
h) 1,2-dihydro-1-(2,3-methylenedioxybenzyl)-7-methyl-4-(4-methoxyphenyl)-2-oxobenzothieno[3,2-b]pyridine-3-carboxylic acid;
i) 1,2-dihydro-1-(2,1,3-benzothiadiazol-5-methyl)-4-(4-methoxyphenyl)-2-oxobenzofuro[3,2-b]pyridine-3-carboxylic acid;
iv) the compounds described in EP 0757039
a) 4-(1,3-benzodioxol-5-yl)-1,2-dihydro-1-(2-methoxybenzyl)-2-oxoquinoline-3-carboxylic acid;
b) 4-(1,3-benzodioxol-5-yl)-1,2-dihydro-1-(4-methoxybenzyl)-2-oxoquinoline-3-carboxylic acid;
c) 4-(1,3-benzodioxol-5-yl)-1,2-dihydro-1-(3,4-methylenedioxybenzyl)-2-oxoquinoline-3-carboxylic acid;
d) 4-(1,3-benzodioxol-5-yl)-1,2-dihydro-1-(2-methoxybenzyl )-2-oxoquinoline-3-acetic acid;
e) 4-(1,3-benzodioxol-5-yl)-1,2-dihydro-1-(3,4-methylenedioxybenzyl)-2-oxoquinoline-3-acetic acid;
f) 4-(1,3-benzodioxol-5-yl)-1,2-dihydro-6-ethoxy-1-(2-methoxybenzyl)-2-oxoquinoline-3-carboxylic acid;
g) 4-(1,3-benzodioxol-5-yl)-1,2-dihydro-6-ethoxy-1-(4-methoxybenzyl)-2-oxoquinoline-3-carboxylic acid;
h) 4-(1,3-benzodioxol-5-yl)-1,2-dihydro-6-ethoxy-1-(6-chloro-3,4-methylenedioxybenzyl)-2-oxoquinoline-3-carboxylic acid;
i) 4-(1,3-benzodioxol-5-yl)-1,2-dihydro-6-ethoxy-1-(3,4-methylenedioxybenzyl)-2-oxoquinoline-3-carboxylic acid;
j) 4-(1,3-benzodioxol-5-yl)-1,2-dihydro-6-ethoxy-1-(3-methoxybenzyl)-2-oxoquinoline-3-carboxylic acid;
v) the compounds described in EP 0796250
a) 2-(1,3-benzodioxol-5-yl)-2-(2,3-dihydro-4,6-dimethylpyridazin-3-on-2-yl)-N-(4-isopropylphenylsulfonyl)acetamide;
b) 2-(1,3-benzodioxol-5-yl)-2-(6-(4-methoxyphenyl)-2,3,4,5-tetrahydropyridazin-3-on-2-yl)-N-(4-isopropylphenylsulfonyl)acetamide;
c) 2-(1,3-benzodioxol-5-yl)-2-(6-(4-chlorophenyl)-2,3,4,5-tetrahydropyridazin-3-on-2-yl)-N-(4-isopropylphenylsulfonyl)acetamide;
d) 2-(1,3-benzodioxol-5-yl)-2-(6-(3,4-dimethoxyphenyl)-2,3,4,5-tetrahydropyridazin-3-on-2-yl)-N-(4-isopropylphenylsulfonyl)acetamide;
e) 2-(1,3-benzodioxol-5-yl)-2-(4-methyl-6-phenyl-2,3-dihydro-pyridazin-3-on-2-yl)-N-(4-isopropylphenylsulfonyl)acetamide;
f) 2-(1,3-benzodioxol-5-yl)-2-(5-(3,4-dimethoxyphenyl)-6-ethyl-2H-3,6-dihydro-1,3,4-thiadiazin-2-on-3-yl)-N-(4-isopropylphenyl-sulfonyl)acetamide;
vi) the compounds described in WO9719077
a) 3-(1,3-benzodioxol-5-yl)-1-(2,1,3-benzothiadiazol-5-ylmethyl)-5-propoxyindole-2-carboxylic acid;
b) 3-(4-methoxyphenyl)-1-(2,1,3-benzothiadiazol-5-ylmethyl)-5-ethoxyindole-2-carboxylic acid;
c) 3-(4-methoxyphenyl)-1-(2,1,3-benzothiadiazol-5-ylmethyl )-5-propoxyindole-2-carboxylic acid;
d) 3-(2,1,3-benzothiadiazol-5-yl)-1-(4-methoxybenzyl )-5-ethoxyindole-2-carboxylic acid;
e) 3-(2,1,3-benzothiadiazol-5-yl)-1-(4-methoxybenzyl)-5-propoxyindole-2-carboxylic acid;
f) 3-(2,1,3-benzothiadiazol-5-yl)-1-(3,4-methylenedioxybenzyl)-5,6-dimethoxyindole-2-carboxylic acid;
vii) the compounds described in WO 9730982
2-(2,1,3-benzothiadiazol-5-yl)-3-benzyl-4-(4-methoxyphenyl)-4-oxo-2-butenoic acid;
2-(2,1,3-benzothiadiazol-5-yl)-3-(3,4,5-trimethoxybenzyl)-4-(4-methoxyphenyl)-4-oxo-2-butenoic acid;
2-(2,1,3-benzothiadiazol-5-yl)-3-(3,4-diisopropoxy-5-methoxybenzyl)-4-(4-methoxyphenyl)-4-oxo-2-butenoic acid;
2-(2,1,3-benzothiadiazol-5-yl)-3-benzyl-4-(1,4-benzodioxan-6-yl )-4-oxo-2-butenoic acid;
2-(2,1,3-benzothiadiazol-5-yl)-3-(3,4,5-trimethoxybenzyl)-4-(1,4-benzodi-oxan-6-yl)-4-oxo-2-butenoic acid;
2-(2,1,3-benzothiadiazol-5-yl)-3-(3,4-diisopropoxy-5-methoxybenzyl)-4-(1,4-benzodioxan-6-yl)-4-oxo-2-butenoic acid;
2-(2,1,3-benzothiadiazol-5-yl)-3-(3,4,5-trimethoxybenzyl)-4-(1,3-benzodioxol-5-yl)-4-oxo-2-butenoic acid;
3-(2,1,3-benzothiadiazol-5-yl)-4-benzyl-5-hydroxy-5-(3-fluoro-4-methoxyphenyl)-5H-furan-2-one;
2-(2,1,3-benzothiadiazol-5-yl)-3-(3,4,5-trimethoxybenzyl)-4-(3-fluoro-4-methoxyphenyl)-4-oxo-2-butenoic acid;
3-(2,1,3-benzothiadiazol-5-yl)-4-[(7-methoxy-1,3-benzodioxol-5-yl)methyl]-5-hydroxy-5-(4-methoxyphenyl)-5H-furan-2-one;
3-(2,1,3-benzothiadiazol-5-yl)-4-(3,4,5-trimethoxybenzyl)-5-hydroxy-5-(3-methoxyphenyl)-5H-furan-2-one;
3-(2,1,3-benzothiadiazoi-5-yl)-4-(3,4,5-trimethoxybenzyl)-5-hydroxy-5-(2-methoxyphenyl)-5H-furan-2-one;
3-(2,1,3-benzothiadiazol-5-yl)-4-(4-methylthiobenzyl )-5-hydroxy-5-(4-methoxyphenyl)-5H-furan-2-one;
3-(2,1,3-benzothiadiazol-5-yl )-4-(3-benzyloxy-4-methoxybenzyl )-5-hydroxy-5-(4-methoxyphenyl)-5H-furan-2-one;
3-(2,1,3-benzothiadiazol-5-yl )-4-(2,3-dihydrobenzofuran-5-ylmethyl )-5-hydroxy-5-(4-methoxyphenyl)-5H-furan-2-one;
3-(2,1,3-benzothiadiazol-5-yl)-4-(2-methylpropyl)-5-hydroxy-5-(4-methoxyphenyl)-5H-furan-2-one;
3-(2,1,3-benzothiadiazol-5-yl)-4-(3,5-dimethoxybenzyl)-5-hydroxy-5-(4-methoxyphenyl)-5H-furan-2-one;
3-(2,1,3-benzothiadiazol-5-yl)-4-(4-tert-butoxybenzyl)-5-hydroxy-5-(4-methoxyphenyl)-5H-furan-2-one;
3-(2,1, 3-benzothiadiazol-5-yl )-4-(4-hydroxybenzyl )-5-hydroxy-5-(4-methoxyphenyl)-5H-furan-2-one;
3-(2,1, 3-benzothiadiazol-5-yl)-4-(4-trifluoromethoxybenzyl )-5-hydroxy-5-(4-methoxyphenyl)-5H-furan-2-one;
3-(2,1,3-benzothiadiazol-5-yl)-4-(3,5-dimethoxy-4-isopropoxybenzyl)-5-hydroxy-5-(4-methoxyphenyl)-5H-furan-2-one;
3-(2,1,3-benzothiadiazol-5-yl)-4-(3,5-dimethoxy-4-pentyloxybenzyl)-5-hydroxy-5-(4-methoxyphenyl)-5H-furan-2-one;
3-(2,1,3-benzothiadiazol-5-yl)-4-(3,5-dimethoxy-4-hexyloxybenzyl)-5-hydroxy-5-(4-methoxyphenyl)-5H-furan-2-one;
3-(2,1,3-benzothiadiazol-5-yl)-4-(4-phenoxybenzyl )-5-hydroxy-5-(4-methoxyphenyl)-5H-furan-2-one;
3-(2,1,3-benzothiadiazol-5-yl)-4-(4,5-dimethoxy-3-isopropoxybenzyl )-5-hydroxy-5-(4-methoxyphenyl)-5H-furan-2-one;
3-(2,1,3-benzothiadiazol-5-yl)-4-(3,4,5-trimethoxybenzyl)-5-hydroxy-5-(2,5-dimethoxyphenyl)-5H-furan-2-one;
3-(2,1,3-benzothiadiazol-5-yl)-4-(3,4,5-trimethoxybenzyl)-5-hydroxy-5-(3-chloro-4-methoxyphenyl)-5H-furan-2-one;
3-(2,1,3-benzothiadiazol-5-yl)-4-(3,4,5-trimethoxybenzyl)-5-hydroxy-5-(3-methyl-4-methoxyphenyl)-5H-furan-2-one;
3-(2,1,3-benzothiadiazol-5-yl )-⁴-(3,⁴-diisopropoxy-5-methoxybenzyl )-5-hydroxy-5-(2,5-dimethoxyphenyl )-5H-furan-2-one;
3-(2,1,3-benzothiadiazol-5-yl)-4-(3,4,5-trimethoxybenzyl)-5-hydroxy-5-(2,3-dihydrobenzofuran-5-yl)-5H-furan-2-one;
3-(2,1,3-benzothiadiazol-5-yl)-4-(3,5-dimethoxy-4-isopropoxybenzyl)-5-hydroxy-5-(3-fluoro-4-methoxyphenyl)-5H-furan-2-one;
3-(2,1,3-benzothiadiazol-5-yl)-4-(3,4-dimethoxy-5-propoxybenzyl)-5-hydroxy-5-(4-methoxyphenyl)-5H-furan-2-one;
3-(2,1,3-benzothiadiazol-5-)-4-(3,4-diisopropoxy-5-methoxybenzyl)-5-hydroxy-5-(4-propoxyphenyl)-5H-furan-2-one;
3-(2,1,3-benzothiadiazol-5-yl )-4-(3,4-diisopropoxy-5-methoxybenzyl )-5-hydroxy-5-(2,4-dimethoxyphenyl)-5H-furan-2-one;
3-(2,1,3-benzothiadiazol-5-yl )-4-(4-benzyloxy-2-methoxybenzyl )-5-hydroxy-5-(4-methoxyphenyl)-5H-furan-2-one;
3-(2,1,3-benzothiadiazol-5-yl)-4-(2,3,4-trimethoxybenzyl)-5-hydroxy-5-(4-methoxyphenyl)-5H-furan-2-one;
3-(2,1,3-benzothiadiazol-5-yl)-4-(3,4, 5-trimethoxybenzyl )-5-hydroxy-5-(2,4-dimethoxyphenyl)-5H-furan-2-one;
3-(2,1,3-benzothiadiazol-5-yl)-4-(3,4,5-triethoxybenzyl )-5-hydroxy-5-(4-methoxyphenyl)-5H-furan-2-one;
3-(2,1,3-benzothiadiazol-5-yl )-4-(3,4,5-trimethoxybenzyl )-5-hydroxy-5-(4-difluoromethoxyphenyl)-5H-furan-2-one;
3-(2,1,3-benzothiadiazol-5-yl )-4-(3-hydroxy-4-methoxybenzyl )-5-hydroxy-5-(4-methoxyphenyl)-5H-furan-2-one;
3-(2,1,3-benzothiadiazol-5-yl)-4-(2,4-dimethoxybenzyl)-5-hydroxy-5-(4-methoxyphenyl)-5H-furan-2-one;
3-(2,1,3-benzothiadiazol-5-yl)-4-(2,4,5-trimethoxybenzyl)-5-hydroxy-5-(4-methoxyphenyl)-5H-furan-2-one;
3-(2,1,3-benzothiadiazol-5-yl )-4-(3,4,5-trimethoxybenzyl)-5-hydroxy-5-(3-fluoro-4-isopropoxyphenyl)-5H-furan-2-one;
3-(2,1,3-benzothiadiazol-5-yl)-4-(3,4,5-trimethoxybenzyl)-5-hydroxy-5-(3-fluoro-4-propoxyphenyl)-5H-furan-2-one;
3-(2,1,3-benzothiadiazol-6-methyl-5-yl)-4-(3,5-dimethoxy-4-isopropoxybenzyl)-5-hydroxy-5-(4-methoxyphenyl )-5H-furna-2-one;
3-(2,1,3-benzothiadiazol-5-yl)-4-(3,5-dimethoxy-4-benzyloxybenzyl)-5-hydroxy-5-(4-methoxyphenyl)-5H-furan-2-one;
3-(2,1,3-benzothiadiazol-5-yl)-4-(3,5-dimethoxy-4-hydroxybenzyl)-5-hydroxy-5-(4-methoxyphenyl)-5H-furan-2-one;
3-(2,1,3-benzothiadiazol-5-yl)-4-(3,5-dimethoxy-4-propoxybenzyl)-5-hydroxy-5-(4-methoxyphenyl)-5H-furan-2-one;
3-(2,1,3-benzothiadiazol-5-yl)-4-(3,4-dimethoxy-5-isopropoxybenzyl)-5-hydroxy-5-(1,4-benzodioxan-6-yl)-5H-furan-2-one;
3-(2,1,3-benzothiadiazol-6-methyl-5-yl)-4-(3,4,5-trimethoxybenzyl )-5-hydroxy-5-(4-methoxyphenyl)-5H-furan-2-one;
3-(2,1,3-benzothiadiazol-5-yl)-4-(4-isopropoxybenzyl)-5-hydroxy-5-(4-methoxyphenyl)-5H-furan-2-one;
3-(2,1,3-benzothiadiazol-5-yl)-4-(4-hexyloxybenzyl )-5-hydroxy-5-(4-methoxyphenyl)-5H-furan-2-one;
3-(2,1,3-benzothiadiazol-5-y)-4-(3,5-dimethoxy-4-isopropoxybenzyl )-5-hydroxy-5-(1,4-benzodioxan-6-yl)-5H-furan-2-one;
3-(2,1,3-benzothiadiazol-5-yl )-4-(3-methoxy-5-butoxybenzyl )-5-hydroxy-5-(4-methoxyphenyl)-5H-furan-2-one;
3-(2,1,3-benzothiadiazol-5-yl)-4-(3,4-diisopropoxybenzyl)-5-hydroxy-5-(4-methoxyphenyl)-5H-furan-2-one;
3-(2,1,3-benzothiadiazol-5-yl)-4-(3,4,5-trimethoxybenzyl )-5-hydroxy-5-(2-fluoro-4-methoxyphenyl)-5H-furan-2-one;
3-(2,1,3-benzothiadiazol-5-yl)-4-(3,4-dimethoxy-5-isopropoxybenzyl)-5-hydroxy-5-(3-fluoro-4-methoxyphenyl)-5H-furan-2-one;
3-(2,1,3-benzothiadiazol-5-yl)-4-(3,4-dimethoxy-5-benzyloxybenzyl)-5-hydroxy-5-(4-methoxyphenyl)-5H-furan-2-one;
3-(2,1,3-benzothiadiazol-5-yl )-4-(3,4, 5-trimethoxybenzyl)-5-hydroxy-5-(4-fluoro-2-methoxyphenyl)-5H-furan-2-one;
3-(2,1,3-benzothiadiazol-5-yl)-4-(3,5-dimethoxy-5-ethoxybenzyl)-5-hydroxy-5-(4-methoxyphenyl)-5H-furan-2-one;
3-(2,1,3-benzothiadiazol-5-yl )-4-(4-methoxycarbonylbenzyl)-5-hydroxy-5-(4-methoxyphenyl)-5H-furan-2-one;
3-(2,1,3-benzothiadiazol-5-yl)-4-(3,4-diisopropoxybenzyl)-5-hydroxy-5-(3-fluoro-4-methoxyphenyl)-5H-furan-2-one;
3-(2,1,3-benzothiadiazol-5-yl)-4-(3,4,5-trimethoxybehzyl)-5-hydroxy-5-(4-benzyloxyphenyl)-5H-furan-2-one;
3-(2,1,3-benzothiadiazol-4-methyl-5-yl)-4-(3,4,5-trimethoxybenzyl)-5-hydroxy-5-(4-methoxyphenyl)-5H-furan-2-one;
3-(2,1,3,-benzothiadiazol-5-yl)-4-(3,5-dimethoxy-4-isobutoxybenzyl)-5-hydroxy-5-(4-methoxyphenyl )-5H-furan-2-one;
4-(2,1,3-benzothiadiazol-5-ylmethyl)-3-(7-methoxy-1, 3-benzodioxol-5-yl)-5-hydroxy-5-(4-methoxyphenyl )-5H-furan-2-one;
2-(2,1,3-benzothiadiazol-5-yl)-3-(3,4-diisopropoxy-5-methoxybenzyl )-4-(3-fluoro-4-methoxyphenyl)-4-oxo-2-butenoic acid;
2-(2,1,3-benzothiadiazol(-5-yl)-3-(3,5-dimethoxy-4-isopropoxybenzyl )-4-(4-methoxyphenyl)-4-oxo-2-butenoic acid;
2-(2,1,3-benzothiadiazol-5-y)-3-(3,4-dimethoxy-5-isopropoxybenzyl )-4-(4-methoxyphenyl)-4-oxo-2-butenoic acid;
2-(2,1,3-benzothiadiazol-5-yl)-3-(3,5-dimethoxy-4-isopropoxybenzyl )-4-(3-fluoro-4-methoxyphenyl)-4-oxo-2-butenoic acid;
2-(2,1,3-benzothiadiazol-5-yl)-3-(3,4,5-trimethoxybenzyl)-4-(4-methoxyphenyl)-4-oxo-2-butenoic acid;
viii) the compounds described in WO 9730996
a) 3-(2,1,3-benzothiadiazol-5-aminosulfonyl)-N-(6-methyl-1,3-benzodioxol-5-yl)thiophene-2-carboxamide;
b) 3-(2,1,3-benzothiadiazol-5-aminosulfonyl)-N-(6-acetyl-1,3-benzodioxol-5-yl)thiophene-2-carboxamide;
c) 3-(2,1,3-benzothiadiazol-5-aminosulfonyl)-N-(6-cyano-1,3-benzodioxol-5-yl)thiophene-2-carboxamide;
d) 3-(2,1,3-benzothiadiazol-5-aminosulfonyl)-2-(6-methyl-1,3-benzodioxol-5-ylmethylcarbonyl)thiophene;
ix) the compounds described in DE 19609597
a) N-(2,1,3-benzothiadiazol-5-yl)-5-N′-isopropylamino-1-naphthalenesulfon-amide;
b) N-(2,1,3-benzothiadiazol-5-yl)-5-N′-propylamino-1-naphthalenesulfon-amide;
c) N-(2,1,3-benzothiadiazol-5-yl)-5-N′-methylamino-1-naphthalenesulfon-amide;
d) N-(2,1,3-benzothiadiazol-5-yl)-5-N′-ethylamino-1-naphthalenesulfon-amide;
e) N-(2,1,3-benzothiadiazol-5-yl)-5-N′-butylamino-1-naphthalenesulfon-amide;
x) the compounds described in DE 19612101
a) 4-(4-methoxyphenyl)-1,6-dihydro-1-(2-methoxybenzyl)-2-methyl-6-oxopyrimidine-5-carboxylic acid;
b) 4-(3,4-methylenedioxyphenyl)-1,6-dihydro-1-(2-methoxybenzyl)-2-cyclopropyl-6-oxopyrimidine-5-carboxylicacid;
c) 4-(2-carboxy-4-methoxy-7-benzofuranyl)-1,6-dihydro-1-(2-methoxybenzyl)-2-methyl-6-oxopyrimidine-5-carboxylicacid;
d) 4-(2-phenyl-4-methoxyphenyl)-1,6-dihydro-1-(2-methoxybenzyl)-2-methyl-6-oxopyrimidine-5-carboxylic acid;
e) 4-(2-carboxy-4-methoxy-7-benzofuranyl)-1,6-dihydro-1-(5-benzothiadiazolyl)-2-methyl-6-oxopyrimidine-5-carboxylic acid;
f) 4-(4-methoxyphenyl)-1,6-dihydro-1-(5-benzothiadiazolyl)-2-methyl-6-oxopyrimidine-5-carboxylic acid;
xi) the compounds described in WO 9827091
a) 4-(2,1,3-benzothiadiazol-5-ylmethyl)-1-benzyl-3-butyl-1H-pyrazole-5-carboxylic acid;
b) 4-(2,1,3-benzothiadiazol-5-ylmethyl)-1-(3-methoxybenzyl )-3-butyl-1H-pyrazole-5-carboxylic acid;
c) 4-(2,1,3-benzothiadiazol-6-chloro-5-ylmethyl )-1-(3-methoxybenzyl)-3-butyl-1H-pyrazole-5-carboxylic acid;
d) 4-(2,1,3-benzothiadiazol-5-ylmethyl )-1-(2-carboxymethoxy-4-methoxybenzyl)-3-butyl-1H-pyrazole-5-carboxylic acid;
e) 4-(2,1,3-benzothiadiazol-5-ylmethyl)-1-(2,4-dimethoxybenzyl)-3-butyl-1H-pyrazole-5-carboxylic acid;
f) 4-(2,1,3-benzothiadiazol-5-ylmethyl)-1-(3-methoxybenzyl)-3-phenyl-1H-pyrazole-5-carboxylic acid;
g) 4-(2,1,3-benzothiadiazol-5-ylmethyl)-1-(3-methoxybenzyl)-3-(2-thienyl)-1H-pyrazole-5-carboxylic acid;
h) 4-(2,1,3-benzothiadiazol-5-ylmethyl)-1-(3-methoxybenzyl)-3-cyclohexyl-1H-pyrazole-5-carboxylic acid;
i) 4-(2,1,3-benzothiadiazol-5-yl methyl)-1-(2-carboxymethoxy-4-methoxybenzyl)-3-propoxy-1H-pyrazole-5-carboxylic acid;
xii) the compounds described in WO 9827077
a) 2-(2,1,3-benzothiadiazol-5-yl )-3-(thien-2-ylmethyl )-4-(4-methoxyphenyl)-4-oxo-2-butenoic acid;
b) 2-(2,1,3-benzothiadiazoi-5-yl)-3-(5-methoxythien-2-ylmethyl)-4-(4-methoxyphenyl)-4-oxo-2-butenoic acid;
c) 3-(2,1,3-benzothiadiazol-5-yl )-4-(furan-2-yl methyl )-5-hydroxy-5-(4-methoxyphenyl)-5H-furan-2-one;
d) 3-(2,1,3-benzothiadiazol-5-yl)-4-(3,4,5-trimethoxybenzyl)-5-hydroxy-5-(3,4-dihydro-2H-1,5-benzodioxepin-7-yl)-5H-furan-2-one;
e) 3-(2,1,3-benzothiadiazol-5-yl)-4-(3,4-diisopropoxy-5-methoxybenzyl)-5-hydroxy-5-(3,4-dihydro-2H-1,5-benzodioxepin-7-yl)-5H-furan-2-one;
f) 3-(2,1,3-benzothiadiazol-5-yl)-4-(thien-3-ylmethyl)-5-hydroxy-5-(4-methoxyphenyl)-5H-furan-2-one;
xiii) the compounds described in WO 9841515
a) 2-(2,1,3-benzothiadiazol-5-yl)-4-(4-methoxyphenyl)-4-oxo-2-butanoic acid;
b) 2-(2,1,3-benzothiadiazol-5-yl)-3-(2,1,3-benzothiadiazol-5-ylmethyl)acetic acid;
c) 2-(2,1,3-benzothiadiazol-5-yl)-2-(4-methoxycarbonylbenzyl)acetic acid;
d) 2-(2,1,3-benzothiadiazol-5-yl )-2-(4-methoxycarbonylbenzyl)-N-(4-isopropylphenylsulfonyl)acetamide;
e) 2-(2,1,3-benzothiadiazol-5-yl)-2-(4-carboxybenzyl)-N-(4-isopropylphenylsulfonyl)acetamide;
f) 2-(2,1,3-benzothiadiazol-5-yl )-4-(4-methoxybenzyl )acetic acid;
g) 2-(2,1,3-benzothiadiazol-5-yl )-2-(4-methoxybenzyl )-4-(4-methoxyphenyl)-4-oxo-2-butanoic acid;
xiv) the compounds described in WO 9841521
a) 2-(1,3-benzodioxol-5-yl)-3-(2,1,3-benzothiadiazol-5-yl)succinic acid;
b) 2,3-bis(1,3-benzodioxol-5-yl)maleic acid;
c) N,N-dibutyl-2,3-bis(1,3-benzodioxol-5-yl)maleamide;
d) 2,3-bis(1,3-benzodioxol-5-yl)maleic anhydride;
e) 2-(1,3-benzodioxol-5-yl)-3-phenylmaleic anhydride;
xv) the compounds described in WO 9842702
ethyl [3-(2,1, 3-benzothiadiazol-5-yl )-4-(3,4,5-trimethoxybenzyl )-5-(4-methoxyphenyl)-5H-furan-2-on-5-yloxycarbonylamino]acetate;
ethyl [3-(2,1,3-benzothiadiazol-5-yl)-4-(3,4,5-trimethoxybenzyl)-5-(3-fluoro-4-methoxyphenyl)-5H-furan-2-on-5-yloxycarbonylamino]acetate;
N-1-naphthylethyl-[3-(2,1,3-benzothiadiazol-5-yl)-4-(3,4,5-trimethoxybenzyl)-5-(3-fluoro-4-methoxyphenyl)-5H-furan-2-on-5-yl]carbamate;
ethyl 2-[3-(2,1,3-benzothiadiazol-5-yl)-4-(3,4,5-trimethoxybenzyl)-5-(3-fluoro-4-methoxyphenyl)-5H-furan-2-on-5-yloxycarbonylamino]-3-methylbutyrate;
2-(2,1,3-benzothiadiazol-5-yl)-3-(3-fluoro-4-methoxybenzoyl)-4-(3,4,5-trimethoxyphenyl)but-2-enoic acid;
3-(2,1,3-benzothiadiazol-5-yl)-4-benzyl-5-hydroxy-5-(3-fluoro-4-methoxyphenyl)-5H-furan-2-one;
3-(2,1, 3-benzothiadiazol-5-yl)-4-(3,4,5-trimethoxybenzyl )-5-hydroxy-5-(3-fluoro-4-methoxyphenyl)-5H-furan-2-one;
3-(2,1,3-benzothiadiazol-5-yl)-4-[(7-methoxy-1,3-benzodioxol-5-yl)methyl]-5-hydroxy-5-(4-methoxyphenyl)-5H-furan-2-one;
3-(2,1,3-benzothiadiazol-5-yl)-4-(3,4,5-trimethoxybenzyl)-5-hydroxy-5-(3-methoxyphenyl)-5H-furan-2-one;
3-(2,1,3-benzothiadiazol-5-yl)-4-(3,4,5-trimethoxybenzyl )-5-hydroxy-5-(2-methoxyphenyl)-5H-furan-2-one;
3-(2,1,3-benzothiadiazol-5-yl)-4-(4-methylthiobenzyl)-5-hydroxy-5-(4-methoxyphenyl)-5H-furan-2-one;
3-(2,1,3-benzothiadiazol-5-yl)-4-(3-benzyloxy-4-methoxybenzyl)-5-hydroxy-5-(4-methoxyphenyl)-5H-furan-2-one;
3-(2,1,3-benzothiadiazol-5-yl )-4-(2,3-dihydrobenzofuran-5-ylmethyl)-5-hydroxy-5-(4-methoxyphenyl)-5H-furan-2-one;
3-(2,1,3-benzothiadiazol-5-yl)-4-(2-methylpropyl)-5-hydroxy-5-(4-methoxyphenyl)-5H-furan-2-one;
3-(2,1,3-benzothiadiazol-5-yl)-4-(3,5-d imethoxybenzyl )-5-hydroxy-5-(4-methoxyphenyl)-5H-furan-2-one;
3-(2,1,3-benzothiadiazol-5-yl )-4-(4-tert-butoxybenzyl )-5-hydroxy-5-(4-methoxyphenyl)-5H-furan-2-one;
3-(2,1,3-benzothiadiazol-5-yl)-4-(4-hydroxybenzyl )-5-hydroxy-5-(4-methoxyphenyl)-5H-furan-2-one;
3-(2,1,3-benzothiadiazol-5-yl)-4-(4-trifluoromethoxybenzyl)-5-hydroxy-5-(4-methoxyphenyl)-5H-furan-2-one;
3-(2,1,3-benzothiadiazol-5-yl)-4-(3,5-dimethoxy-4-isopropoxybenzyl)-5-hydroxy-5-(4-methoxyphenyl )-5H-furan-2-one;
3-(2,1,3-benzothiadiazol-5-yl)-4-(3,5-dimethoxy-4-pentyloxybenzyl)-5-hydroxy-5-(4-methoxyphenyl)-5H-furan-2-one;
3-(2,1,3-benzothiadiazol-5-yl )-4-(3,5-dimethoxy-4-hexyloxybenzyl )-5-hydroxy-5-(4-methoxyphenyl)-5H-furan-2-one;
3-(2,1,3-benzothiadiazol-5-yl)-4-(4-phenoxybenzyl)-5-hydroxy-5-(4-methoxyphenyl)-5H-furan-2-one;
3-(2,1,3-benzothiadiazol-5-yl)-4-(4,5-dimethoxy-3-isopropoxybenzyl)-5-hydroxy-5-(4-methoxyphenyl)-5H-furan-2-one;
3-(2,1,3-benzothiadiazol-5-yl)-4-(3,4,5-trimethoxybenzyl)-5-hydroxy-5-(2,5-dimethoxyphenyl)-5H-furan-2-one;
3-(2,1,3-benzothiadiazol-5-yl)-4-(3,4,5-trimethoxybenzyl)-5-hydroxy-5-(3-chloro-4-methoxyphenyl)-5H-furan-2-one;
3-(2,1,3-benzothiadiazol-5-yl)-4-(3,4,5-trimethoxybenzyl)-5-hydroxy-5-(3-methyl-4-methoxyphenyl)-5H-furan-2-one;
3-(2,1,3-benzothiadiazol-5-yl)-4-(3,4-diisopropoxy-5-methoxybenzyl)-5-hydroxy-5-(2,5-dimethoxyphenyl)-5H-furan-2-one;
3-(2,1,3-benzothiadiazol-5-yl)-4-(3,4, 5-trimethoxybenzyl)-5-hydroxy-5-(2,3-dihydrobenzofuran-5-yl)-5H-furan-2-one;
3-(2,1,3-benzothiadiazol-5-yl)-4-(3,5-dimethoxy-4-isopropoxybenzyl)-5-hydroxy-5-(3-fluoro-4-methoxyphenyl)-5H-furan-2-one;
3-(2,1,3-benzothiadiazol-5-yl)-4-(3,4-dimethoxy-5-propoxybenzyl)-5-hydroxy-5-(4-methoxyphenyl)-5H-furan-2-one;
3-(2,1,3-benzothiadiazol-5-yl)-4-(3,4-diisopropoxy-5-methoxybenzyl)-5-hydroxy-5-(4-propoxyphenyl)-5H-furan-2-one;
3-(2,1,3-benzothiadiazol-5-yl)-4-(3,4-diisopropoxy-5-methoxybenzyl)-5-hydroxy-5-(2,4-dimethoxyphenyl)-5H-furan-2-one;
3-(2,1,3-benzothiadiazol-⁵-yl)-4-(4-benzyloxy-2-methoxybenzyl )-5-hydroxy-5-(4-methoxyphenyl)-5H-furan-2-one;
3-(2,1, 3-benzothiadiazol-5-yl)-4-(2,3,4-trimethoxybenzyl )-5-hydroxy-5-(4-methoxyphenyl)-5H-furan-2-one;
3-(2,1,3-benzothiadiazol-5-yl)-4-(3,4,5-trimethoxybenzyl)-5-hydroxy-5-(2,4-dimethoxyphenyl)-5H-furan-2-one;
3-(2,1,3-benzothiadiazol-5-yl )-4-(3,4,5-triethoxybenzyl )-5-hydroxy-5-(4-methoxyphenyl)-5H-furan-2-one;
3-(2,1,3-benzothiadiazol-5-yl )-4-(3,4,5-trimethoxybenzyl )-5-hydroxy-5-(4-difluoromethoxyphenyl)-5H-furan-2-one;
3-(2,1,3-benzothiadiazol-5-yl )-4-(3-hydroxy-4-methoxybenzyl )-5-hydroxy-5-(4-methoxyphenyl)-5H-furan-2-one;
3-(2,1,3-benzothiadiazol-5-yl )-4-(2,4-dimethoxybenzyl )-5-hydroxy-5-(4-methoxyphenyl)-5H-furan-2-one;
3-(2,1,3-benzothiadiazol-5-yl)-4-(2,4,5-trimethoxybenzyl)-5-hydroxy-5-(4-methoxyphenyl)-5H-furan-2-one;
3-(2,1,3-benzothiadiazol-5-yl)-4-(3,4,5-trimethoxybenzyl )-5-hydroxy-5-(3-fluoro-4-isopropoxyphenyl)-5H-furan-2-one;
3-(2,1,3-benzothiadiazol-5-yl)-4-(3,4,5-trimethoxybenzyl)-5-hydroxy-5-(3-fluoro-4-propoxyphenyl)-5H-furan-2-one;
3-(2,1,3-benzothiadiazol-6-methyl-5-yl)-4-(3,5-dimethoxy-4-isopropoxybenzyl)-5-hydroxy-5-(4-methoxyphenyl)-5H-furan-2-one;
3-(2,1,3-benzothiadiazol-5-yl)-4-(3,5-dimethoxy-4-benzyloxybenzyl)-5-hydroxy-5-(4-methoxyphenyl)-5H-furan-2-one;
3-(2,1,3-benzothiadiazol-5-yl)-4-(3,5-dimethoxy-4-hydroxybenzyl)-5-hydroxy-5-(4-methoxyphenyl)-5H-furan-2-one;
3-(2,1,3-benzothiadiazol-5-yl)-4-(3,5-dimethoxy-4-propoxybenzyl)-5-hydroxy-5-(4-methoxyphenyl)-5H-furan-2-one;
3-(2,1,3-benzothiadian)-5-yl)-4-(3,4-dimethoxy-5-isopropoxybenzyl)-5-hydroxy-5-(1,4-benzodioxan-6-yl)-5H-furna-2-one;
3-(2,1,3-benzothiadiazol-6-methyl-5-yl)-4-(3,4,5-trimethoxybenzyl )-5-hydroxy-5-(4-methoxyphenyl)-5H-furan-2-one;
3-(2,1,3-benzothiadiazol-5-yl)-4-(4-isopropoxybenzyl)-5-hydroxy-5-(4-methoxyphenyl)-5H-furan-2-one;
3-(2,1,3-benzothiadiazol-5-yl)-4-(4-hexyloxybenzyl)-5-hydroy-5-(4-methoxyphenyl)-5H-furan-2-one;
3-(2,1,3-benzothiadiazol-5-yl )-4-(3,5-dimethoxy-4-isopropoxybenzyl)-5-hydroxy-5-(1,4-benzodioxan-6-yl)-5H-furan-2-one;
3-(2,1,3-benzothiadiazol-5-yl)-4-(3-methoxy-5-butbxybenzyl)-5-hydroxy-5-(4-methoxyphenyl)-5H-furan-2-one;
3-(2,1,3-benzothiadiazol-5-yl)-4-(3,4-diisopropoxybenzyl )-5-hydroxy-5-(4-methoxyphenyl)-5H-furan-2-one;
3-(2,1,3-benzothiadiazol-5-yl)-4-(3,4,5-trimethoxybenzyl)-5-hydroxy-5-(2-fluoro-4-methoxyphenyl)-5H-furan-2-one;
3-(2,1,3-benzothiadiazol-5-yl)-4-(3,4-dimethoxy-5-isopropoxybenzyl)-5-hydroxy-5-(3-fluoro-4-methoxyphenyl)-5H-furan-2-one;
3-(2,1,3-benzothiadiazol-5-yl)-4-(3,4-dimethoxy-5-benzyloxybenzyl)-5-hydroxy-5-(4-methoxyphenyl)-5H-furan-2-one;
3-(2,1,3-benzothiadiazol-5-yl)-4-(3,4,5-trimethoxybenzyl)-5-hydroxy-5-(4-fluoro-2-methoxyphenyl)-5H-furan-2-one;
3-(2,1,3-benzothiadiazol-5-y)-4-(3,5-dimethoxy-5-ethoxybenzyl)-5-hydroxy-5-(4-methoxyphenyl)-5H-furan-2-one;
3-(2,1,3-benzothiadiazol-5-yl)-4-(4-methoxycarbonylbenzyl)-5-hydroxy-5-(4-methoxyphenyl)-5H-furan-2-one;
3-(2,1,3-benzothiadiazol-5-yl)-4-(3,4-diisopropoxybenzyl)-5-hydroxy-5-(3-fluoro-4-methoxyphenyl)-5H-furan-2-one;
3-(2,1,3-benzothiadiazol-5-yl)-4-(3,4,5-trimethoxybenzyl)-5-hydroxy-5-(4-benzyloxyphenyl)-5H-furna-2-one;
3-(2,1,3-benzothiadiazol-4-methyl-5-yl)-4-(3,4,5-trimethoxybenzyl)-5-hydroxy-5-(4-methoxyphenyl)-5H-furan-2-one;
3-(2,1,3-benzothiadiazol-5-yl)-4-(3,5-dimethoxy-4-isobutoxybenzyl)-5-hydroxy-5-(4-methoxyphenyl)-5H-furan-2-one;
4-(2,1,3-benzothiadiazol-5-yl )-3-(7-methoxy-1,3-benzodioxol-5-yl)-5-hydroxy-5-(4-methoxyphenyl)-5H-furan-2-one;
and the open-chain tautomers;
xvi) the compounds described in WO 9842709
a) 3-(2,1,3-benzothiadiazol-5-ylmethyl)-1-(4-methoxyphenyl)-8-methyl-3,8-dihydro-3,8-diazacyclopenta[a)indene-2-carboxylic acid;
b) 3-(2-methoxybenzyl)-1-(4-methoxyphenyl)-8-methyl-3,8-dihydro-3,8-diazacyclopenta[a]indene-2-carboxylic acid;
c) 3-(2,5-dimethoxybenzyl)-1-(4-methoxyphenyl)-8-methyl-3,8-dihydro-3,8-diazacyclopenta[a]indene-2-carboxylicacid;
d) 3-(1,3-benzodioxol-5-ylmethyl)-1-(4-methoxyphenyl)-8-methyl-3,8-dihydro-3,8-diazacyclopenta[a]indene-2-carboxylic acid;
e) 3-(2,1,3-benzothiadiazol-5-ylmethyl)-1-(4-methoxyphenyl)-8-oxa-3-azacyclopenta[a]indene-2-carboxylic acid;
f) 3-(2,1,3-benzothiadiazol-5-ylmethyl)-1-(4-methoxyphenyl)-8-thia-3-azacyclopenta[a]indene-2-carboxylic acid;
g) 3-(2,1,3-benzothiadiazol-5-ylmethyl)-1-(3-carboxymethoxy-4-methoxyphenyl)-8-methyl-3,8-dihydro-3,8-diazacyclopenta[a]indene-2-carboxylic acid;
h) 3-(2,1,3-benzothiadiazol-5-ylmethyl )-1-(3-carboxymethoxy-4-methoxyphenyl)-8-oxa-3-azacyclopenta[a]indene-2-carboxylicacid;
i) 3-(2,1,3-benzothiadiazol-5-ylmethyl)-1-(3-carboxymethoxy-4-methoxyphenyl)-8-thia-3-azacyclopenta[a]indene-2-carboxylic acid;
xvii) the compounds described in WO 9905132
a) 2-(2,1,3-benzothiadiazol-5-yl)-3-(4-cyclopentyloxy-3,5-dimethoxybenzyl)-4-(4-methoxyphenyl )-4-oxo-2-butenoic acid;
b) 2-(2,1,3-benzothiadiazol-5-yl )-3-(4-cyclopentyloxy-3,5-dimethoxybenzyl )-4-(3-fluoro-4-methoxyphenyl)-4-oxo-2-butenoic acid;
c) 3-(2,1,3-benzothiadiazol-5-yl )-4-(4-cyclopentyloxy-3,5-dimethoxybenzyl)-5-hydroxy-5-(4-methoxyphenyl)-5H-furan-2-one;
d) 3-(2,1,3-benzothiadiazol-5-yl)-4-(4-cyclopentyloxy-3,5-dimethoxybenzyl )-5-hydroxy-5-(3-fluoro-4-methoxyphenyl)-5H-furan-2-one;
e) 3-(2,1,3-benzothiadiazol-5-yl )-4-(3-cyclopentyloxy-4,5-dimethoxybenzyl )-5-hydroxy-5-(3-fluoro-4-methoxyphenyl)-5H-furan-2-one;
f) 3-(7-methyl-2,1,3-benzothiadiazol-5-yl )-4-(4-cyclopentyloxy-3,5-dimethoxybenzyl)-5-hydroxy-5-(3-fluoro-4-methoxyphenyl)-5H-furan-2-one;
and physiologically acceptable salts and/or solvates thereof for the preparation of a medicament for inhibiting the growth of neoplastic cells.

3. Use of endothelin receptor antagonists selected from the group consisting of
a) 5-dimethylamino-N-(2,1,3-benzothiadiazol-5-yl)naphthalenesulfonamide;
b) 2-(2,1,3-benzothiadiazol-5-yl)-3-(3-fluoro-4-methoxybenzoyl)-4-(3,4,5-trimethoxyphenyl)but-2-enoic acid;
and physiologically acceptable salts and/or solvates thereof for the preparation of a medicament for inhibiting the growth of neoplastic cells.

4. Use of endothelin receptor antagonists as defined in claim 1
for the preparation of a medicament for the treatment and/or prophylaxis of cancer diseases.

5. Use of endothelin receptor antagonists as defined in claim 1
for the preparation of a medicament for the treatment of precancerogenic damage.

6. Use of endothelin receptor antagonists as defined in claim 1
for the preparation of a medicament for regulating apoptosis in human cells.

7. Use according to claim 4, where the cancer diseases are selected from the group consisting of
prostate cancer, ovarian carcinoma, intestinal cancer, cervical carcinoma, melanoma and pancreatic cancer.
